(19) United States
(12) Patent Application Publication
(10) Pub. No.: US 2024/0131365 A1
(43) Pub. Date: Apr. 25, 2024

(54) SONOTRODE

(71) Applicant: Alma Lasers Ltd., Caesarea (IL)

(72) Inventors: Alexander Britva, Migdal Ha'Emek (IL); Alexander Dverin, Netanya (IL); Yevgeny Pens, Kiryat Bialik (IL); Alexander Belozertsev, Petah Tikva (IL)

(21) Appl. No.: 18/403,831

(22) Filed: Jan. 4, 2024

Related U.S. Application Data
(63) Continuation of application No. 17/679,053, filed on Feb. 23, 2022.
Application No. 17/679,053 is a continuation of application No. PCT/IB21/56408, filed on Jul. 15, 2021.
(60) Provisional application No. 63/052,828, filed on Jul. 16, 2020.

(30) Foreign Application Priority Data
Apr. 8, 2021 (GB) 2105076.0

(51) Int. Cl. A61N 7/00

(52) U.S. Cl. CPC A61N 7/00; A61N 2007/0008; A61N 2007/0056

(57) ABSTRACT

Disclosed are devices suitable for treatment of subcutaneous tissue by transdermally-inducing ultrasonic vibrations in subcutaneous tissue and/or transdermally delivering energy with electromagnetic radiation such as light to subcutaneous tissue. In some embodiments, the treatment of subcutaneous tissue is effective in reducing the amount of subcutaneous fat therein. In some embodiments, transdermal radiation-delivery of energy and transdermal induction of ultrasonic vibrations in subcutaneous tissue can be performed simultaneously, alternatingly or in an unrelated fashion. In some embodiments, the device simultaneously transdermally-induces both ultrasonic transverse and ultrasonic longitudinal vibrations in subcutaneous tissue.

### RELATED APPLICATIONS

The present application gains priority from U.S. patent application Ser. No. 17/679,053, filed 23 Feb. 2022, from International Application PCT/IB21/56408 filed 15 Jul. 2021, from U.S. Provisional Patent Application 63/052,828 filed 16 Jul. 2020, and from U.K. Patent Application GB 2105076.0 filed 9 Apr. 2021, all of which are incorporated by reference as if fully set-forth herein.

### FIELD AND BACKGROUND OF THE INVENTION

The invention, in some embodiments, relates to the treatment of body tissue with energy and more particularly, but not exclusively, to devices for treatment of subcutaneous tissue by transdermally-inducing ultrasonic vibrations in subcutaneous tissue and/or transdermally delivering energy with electromagnetic radiation such as light to subcutaneous tissue. In some embodiments, the treatment of the subcutaneous tissue is effective in reducing the amount of subcutaneous fat therein. In some embodiments, transdermal radiation-delivery of energy and transdermal induction of ultrasonic vibrations in subcutaneous tissue can be performed simultaneously, alternatingly or in an unrelated fashion. In some embodiments, the device simultaneously transdermally induces both ultrasonic transverse and longitudinal vibrations in subcutaneous tissue.

In the art it is known to apply ultrasonic vibrations to a skin surface to transdermally induce ultrasonic vibrations to acoustically deliver energy to subcutaneous tissue such as a subcutaneous adipose tissue layer to damage adipocytes, for example in the field of body sculpting.

Application of ultrasonic vibrations to a surface is typically performed by a device 10 (see FIG. 1) that includes an ultrasonic transducer 12 for generation of ultrasonic longitudinal vibrations having a proximal face 14 functionally associated with an acoustic reflector 16 (e.g., a Langevin-type transducer comprising a stack of piezoelectric elements and the acoustic reflector 16 held together by an axial bolt 17) and a distal face 18 and a distal sonotrode 20 having a proximal face 22, a distal end 24 defining a working face 26 of sonotrode 20 that constitutes an acoustic radiative surface and a sonotrode axis 28, where the proximal face 22 of the sonotrode 20 is acoustically coupled to the distal face 18 of the ultrasonic transducer 12. Typically, either or both the acoustic reflector 16 and the sonotrode 20 are at least partially surrounded by a cooling component, e.g., a water-circulation cooling jacket to cool these components during use.

For use, while the working face 26 of the sonotrode 20 is acoustically coupled to a surface 30 of a medium 32 (e.g., by direct contact or by indirect contact through a coupling substance, e.g., a liquid or gel), an alternating current (AC) oscillating at an ultrasonic driving frequency is supplied from an ultrasound power supply 34 to drive the ultrasonic transducer 12. The piezoelectric elements of the ultrasonic transducer 12 expand and relax at the driving frequency in response to the oscillations of the AC potential, thereby generating ultrasonic longitudinal vibrations with the frequency of the driving frequency. The generated ultrasonic longitudinal vibrations propagate in parallel with the axis 28 through the sonotrode 20 from the proximal face 22 of the sonotrode to the working face 26. The working face 26 applies the ultrasonic longitudinal vibrations to the surface 30, inducing ultrasonic longitudinal vibrations in the medium 32.

For practical use it is advantageous to configure a sonotrode to function as an acoustic amplitude transformer that increases the amplitude of the ultrasonic longitudinal vibrations (i.e., the maximal displacement of distal working face 26) from being relatively small at the proximal face 22 of the sonotrode 20 to substantially larger at the working face 26, typically to between 10 and 150 micrometers. Such configuration includes that the total length 36 of the sonotrode (from proximal face 22 to working face 26) is an integral multiple of λ_{longitudinal}/2, λ_{longitudinal} being the wavelength of the ultrasonic longitudinal vibrations in the sonotrode so that the sonotrode functions as a half-wavelength resonator. The exact value of the length λ_{longitudinal}/2 is dependent on the driving frequency and on the longitudinal speed of sound along the axis 28 of the sonotrode 20.

An additional manner to configure a sonotrode to function as an acoustic amplitude transformer is for the sonotrode to distally taper from a large cross section proximal end 22 to a small cross section closer to the working face 26. The most popular such tapered acoustic amplitude transformer configurations are schematically depicted in side cross section in FIG. 2: FIG. 2A a linear taper sonotrode 38a, FIG. 2B an exponential taper sonotrode 38b, and FIG. 2C a stepped taper sonotrode 38c.

When a sonotrode 20, 38a, 38b or 38c, as depicted in FIG. 1, 2A, 2B or 2C respectively is used, the ultrasonic vibrations in the sonotrode and that are induced in a medium 32 are predominantly, if not entirely, longitudinal vibrations that propagate collinearly with the axis 28 of the sonotrode. The biological effects of energy transdermally delivered by ultrasonic longitudinal vibrations primarily arise from heating of tissue, especially heating of the dermis.

In patent publication US 2011/0213279 which is included by reference as if fully set-forth herein, some of the Inventors disclosed a “mushroom-shaped” sonotrode. In FIG. 2D, such a mushroom-shaped sonotrode 38d is schematically depicted in side cross section having a tapering stem 40 that functions as an acoustic amplitude transformer as described above (particularly similar to stepped-taper sonotrode 38c depicted in FIG. 2C) and a broader distal cap 42. Distal cap 42 is lenticular, in side cross section resembling a lens having a curved back side 44 and a convex working face 26. Working face 26 of sonotrode 38d also includes concentric circular transverse-wave transferring ridges 46.

As detailed in US 2011/0213279, a sonotrode such as 38d is operative to transdermally induce, depending on the value of the driving frequency, either ultrasonic longitudinal vibrations or ultrasonic transverse vibrations in subcutaneous tissue when the working face 26 is acoustically coupled with skin.

Without wishing to be held to any one theory, it is currently believed that with some driving frequencies the ultrasonic longitudinal vibrations generated by an ultrasonic transducer 12 preferentially propagate in parallel with the axis 28 of mushroom-shaped sonotrode such as 38d from the proximal face 22 to the working face 26. These ultrasonic longitudinal vibrations primarily lead to ultrasonic longitudinal vibrations of the sonotrode 38d, which are applied by working face 26 to a skin surface acoustically coupled with working face 26, transdermally-inducing ultrasonic longitudinal vibrations in the subcutaneous tissue.

However, with some other different driving frequencies the ultrasonic longitudinal vibrations generated by an ultrasonic transducer 12 preferentially produce ultrasonic shear wave vibrations in the cap 42 of sonotrode 38d, the ultrasonic shear wave vibrations being perpendicular to the longitudinal vibrations in the stem 40, that is to say, a greater proportion of the energy transferred by the transducer 12 into the sonotrode 38d is in ultrasonic shear wave vibrations in the cap 42 perpendicular to axis 28 rather than ultrasonic longitudinal vibrations parallel with axis 28. As a result, working face 26 substantially vibrates transversely, presumably alternately increasing and decreasing in diameter. When the vibrating working face 26 is applied to a skin surface, the ultrasonic shear wave vibrations induce ultrasonic transverse vibrations in the subcutaneous tissue by virtue of the convex shape of working face 26 and by virtue of the concentric circular transverse-wave transferring ridges 46 that can be considered as physically moving the skin and tissue transversely. A device including a sonotrode such as 38d provides two modes of operation:
at a first driving frequency that is related to the wavelength 4 for which the sonotrode 38d is configured to act as an acoustic amplitude transformer, a first “hot” or “longitudinal” mode where the energy transdermally delivered to subcutaneous tissue through the working face 26 is primarily by ultrasonic longitudinal vibrations that are perpendicular to the skin surface; and
at a second driving frequency different from the first driving frequency, a second “cold” or “transverse” mode where the energy transdermally-delivered to subcutaneous tissue through the working face 26 is primarily by ultrasonic transverse vibrations that are parallel to the skin surface. As described in US 2011/0213279, relatively low-energy “cold” ultrasonic transverse waves disrupt adipocytes, apparently by repeatedly stretching the adipocyte cell membranes and then allowing these to relax, yet cause substantially no collateral damage to surrounding non-adipose tissue.

In some preferred embodiments described in US 2011/0213279, ultrasonic longitudinal vibrations of the first mode and ultrasonic shear wave vibrations of the second mode are alternately applied through a mushroom-shaped sonotrode such as 38d. The ultrasonic longitudinal vibrations are applied by the working face 26 to the skin surface (typically for a duration of about 5 seconds) to transdermally-induce ultrasonic longitudinal waves that heat subcutaneous tissue such as the dermis. Subsequently ultrasonic shear wave vibrations are applied by the working face 26 to the skin surface (typically for a duration of about 15 seconds) to induce ultrasonic transverse vibrations to disrupt the adipocytes. Because of the preceding heating by the ultrasonic longitudinal vibrations, the ultrasonic transverse vibrations penetrate more deeply and/or more effectively and/or a greater fraction of the energy penetrates to a given depth of the adipose tissue and/or the heated tissue has improved energy-absorbing properties.

Although highly effective in the field of body sculpting, a sonotrode such as described in US 2011/0213279 is sometimes considered less than ideal for some uses because the shear wave vibrations are not applied continuously, because of the added complexity required for generating and switching between two different driving frequencies and because, if a user moves the working face over different portions of a treated subject too quickly, the results of a treatment might be considered less than ideal.

In patent publication US 2019/0091490 which is included by reference as if fully set-forth herein, some of the Inventors disclose a sonotrode that simultaneously transdermally induces both ultrasonic transverse and ultrasonic longitudinal vibrations in subcutaneous tissue, both modes of vibrations having sufficient intensity to deliver substantial energy to achieve a desired biological effect, e.g., substantial heating of tissue by induced longitudinal vibrations and substantial disrupting of adipocytes by induced transverse vibrations. Further, the energy delivered by each one of the two modes is “balanced”, that is to say, during normal use by a body sculpting technician having ordinary skill in the art, the induced ultrasonic transverse vibrations are sufficiently intense to effectively disrupt adipocytes as described in US 2011/0213279 and the simultaneously-induced ultrasonic longitudinal vibrations are sufficiently intense to heat subcutaneous tissue sufficiently to increase the efficacy of the induced ultrasonic transverse vibrations without being so intense as to easily cause potentially catastrophic overheating of body tissue (e.g., burns, scarring). The Inventors believe that the continuous and simultaneous induction of both transverse and longitudinal vibrations is what leads to the particular efficacy of the sonotrode disclosed in US 2019/0091490, for example for the reduction of fat in subcutaneous tissue.

### SUMMARY OF THE INVENTION

The invention, in some embodiments, relates to the treatment of body tissue with energy and more particularly, but not exclusively, to devices for treatment of subcutaneous tissue by transdermally-inducing ultrasonic vibrations in subcutaneous tissue and/or transdermally delivering energy with electromagnetic radiation such as light to subcutaneous tissue. In some embodiments, the treatment of subcutaneous tissue is effective in reducing the amount of subcutaneous fat therein. In some embodiments, transdermal radiation-delivery of energy and transdermal induction of ultrasonic vibrations in subcutaneous tissue can be performed simultaneously, alternatingly or in an unrelated fashion. In some embodiments, the device simultaneously transdermally-induces both ultrasonic transverse and ultrasonic longitudinal vibrations in subcutaneous tissue.

Device with Sonotrode Having a Conical Portion

According to an aspect of some embodiments of the invention there is provided a device suitable for treating subcutaneous tissue, comprising:
a. an ultrasonic transducer for generation of ultrasonic vibrations having a proximal face and a distal face; and
b. a sonotrode with a sonotrode axis including:
   i. a proximal face in contact with and acoustically-coupled to the distal face of the ultrasonic transducer,
   ii. a conical portion having a smaller-radius proximal end and a larger-radius distal end, wherein the conical portion is defined by a conical wall having an outer conical surface and an inner conical surface, which inner conical surface at least partially defines a hollow, and
   iii. a ring portion extending radially outwards from the distal end of the conical portion having a ring-shaped proximal face and a ring-shaped distal face, said ring-shaped distal face being the working face of the sonotrode, the hole of the working face constituting an open end of the hollow.

In some embodiments, the device is configured to irradiate a skin-surface apparent through the hole of the working face of the sonotrode with electromagnetic radiation. The configuration for irradiation is such that the radiation comes from inside the hollow towards the open end of the hollow. As used herein, a skin-surface apparent through the hole of the working face refers to the area of a skin surface that is encompassed by the hole of the working face of the sonotrode when the working face contacts a skin surface.

In some embodiments, the ultrasonic transducer is a Langevin-type transducer including an axial bolt having a distal end and a proximal end. In some such embodiments, the axial bolt includes an axial passage between the distal end and the proximal end of the bolt. In some such embodiments, the axial passage provides fluid communication (e.g., of air) between the distal end and the proximal end of the bolt. Additionally or alternatively, in some embodiments the axial passage provides optical communication (e.g., of electromagnetic radiation such as light) between the distal end and the proximal end of the bolt. Additionally or alternatively, in some embodiments the axial passage provides for the passage of a physical component (e.g., a waveguide such as light guide for example an optical fiber, a suction conduit, a material-delivery conduit) between the distal end and the proximal end of the bolt.

In some embodiments, the diameter of the hole in the working face is between 10% and 70% of the diameter of the ring portion.

In some embodiments, the sonotrode further comprises a stem, the stem having a proximal face that is the proximal face of the sonotrode and a distal end which is the proximal end of the conical wall.

Device with a Hollow in the Sonotrode

Some embodiments of the invention relate to a hollow sontrode having any shape which has a hollow. Thus, according to an aspect of some embodiments of the invention there is also provided a device suitable for treating subcutaneous tissue, comprising:
a. an ultrasonic transducer for generation of ultrasonic vibrations having a proximal face and a distal face; and
b. a sonotrode with a sonotrode axis including:
   i. a proximal face in contact with and acoustically-coupled to the distal face of the ultrasonic transducer,
   ii. in said sonotrode, an open-ended hollow,
   iii. a distal face, said distal face being the working face of the sonotrode, the hole of the working face constituting an open end of the hollow.
      The hollow sonotrode comprises a sonotrode wall having an outer wall surface and an inner wall surface, which inner wall surface at least partially defines the hollow. In some embodiments, the working face is ring-shaped.

In some such embodiments, the device having a sonotrode with a hollow is configured to irradiate a skin-surface apparent through the hole of the working face of the sonotrode with electromagnetic radiation. The configuration for irradiation is such that the radiation comes from inside the hollow towards the open end of the hollow. The shape of the hollow is any suitable shape. In preferred embodiments, the hollow has a cross sectional area (perpendicular to the sonotrode axis) at the open end of the hollow that is larger than the cross sectional area (perpendicular to the sonotrode axis) at the proximal end of the hollow (near the distal face of transducer), for example, the conical hollow described herein. Such a shape allows a greater surface area of skin to be irradiated at any one moment.

Additionally or alternatively to the configuration for irradiating the skin, in some embodiments, the ultrasonic transducer is a Langevin-type transducer including an axial bolt having a distal end and a proximal end. In some such embodiments, the axial bolt includes an axial passage between the distal end and the proximal end of the bolt. In some such embodiments, the axial bolt includes an axial passage between the distal end and the proximal end of the bolt. In some such embodiments, the axial passage provides fluid communication (e.g., of air) between the distal end and the proximal end of the bolt. Additionally or alternatively, in some embodiments the axial passage provides optical communication (e.g., of electromagnetic radiation such as light) between the distal end and the proximal end of the bolt. Additionally or alternatively, in some embodiments the axial passage provides for the passage of a physical component (e.g., a waveguide such as light guide for example an optical fiber, a suction conduit and/or a material delivery-conduit for delivery of a material such as a medicament or cosmetic treatment composition) between the distal end and the proximal end of the bolt.

### Proximal Channel

In some embodiments, in a device of the teachings herein that comprises a sonotrode having a hollow (whether or not having a conical portion), the sonotrode further comprises a proximal channel between the hollow and the outside of the sonotrode near the proximal end of the sonotrode, e.g., at the proximal face of the sonotrode. In some such embodiments, the proximal channel provides fluid communication (e.g., of air) between the hollow and outside the sonotrode. Additionally or alternatively, in some embodiments the proximal channel provides optical communication (e.g., of electromagnetic radiation such as light) between the hollow and outside the sonotrode. Additionally or alternatively, in some embodiments, the proximal channel provides for the passage of a physical component e.g., a waveguide such) as light guide for example an optical fiber, a suction conduit and/or a material delivery-(conduit between the hollow and outside the sonotrode. In some embodiments, the ultrasonic transducer is a Langevin-type transducer including an axial bolt having an axial passage between a distal end and a proximal end of the axial bolt and the sonotrode comprises a bore for engaging the distal end of the axial bolt, so that the proximal channel of the sonotrode and the axial passage of the axial bolt together provide communication between the hollow of the sonotrode and the proximal end of the axial bolt.

In some embodiments, the communication is fluid communication (e.g., of air) between the hollow and the proximal end of the axial bolt.

Additionally or alternatively, in some embodiments then communication is optical communication (e.g., of electromagnetic radiation such as light) between the hollow and the proximal end of the axial bolt.

Additionally or alternatively, in some such embodiments the communication is provision of a passage of a physical component e.g., a waveguide such as light guide for) (example an optical fiber, a suction conduit and/or a material delivery-conduit between the hollow and the proximal end of the axial bolt.

### Non-Axial Through Channel

In some embodiments, in a device of the teachings herein that comprises a sonotrode having a hollow (whether or not having a conical portion, whether or not having communication between the hollow and the proximal end of the axial bolt), the sonotrode comprises a non-axial through-channel between the hollow and outside of the sonotrode through the wall which inner surface defines the hollow (e.g., in some embodiments the conical wall) and/or a stem if present. In some embodiments, the non-axial through-channel provides fluid communication (e.g., of air) between the hollow and the outside. Additionally or alternatively, in some embodiments the non-axial through-channel provides optical communication (e.g., of electromagnetic radiation such as light) between the hollow and the outside. Additionally or alternatively, in some such embodiments the non-axial through-channel provides for the passage of a physical component e.g., a waveguide such as light) (guide for example an optical fiber, a suction conduit, a material-delivery conduit between the hollow and the outside.

### Application of Suction

In some embodiments, in a device of the teachings herein that comprises a sonotrode having a hollow (whether or not having a conical portion) is configured to apply suction to a skin-surface apparent through the hole of the working face of the sonotrode. In some such embodiments, the device is functionally-associated with a suction generator (e.g., a vacuum pump) and a conduit providing fluid communication between the hollow and the suction generator so that activation of the suction generator leads to evacuation of air from the hollow through the channel when the working face contacts a skin surface, the evacuation of air from the hollow by a suction generator leads to a partial vacuum in the hollow thereby applying suction to a skin surface apparent through the hole. In some embodiments, the functionally-associated suction generator and/or conduit are components of the device. Alternatively, in some embodiments, the functionally-associated suction generator and/or the conduit are not components of the device.

In some such embodiments, the device is configured to allow application of suction to the skin surface apparent through the hole of the working face simultaneously with activation of the transducer to induce ultrasonic vibrations in subcutaneous tissue.

Additionally or alternatively, in some such embodiments, the device is configured to allow application of suction to the skin surface apparent through the hole of the working face alternating with activation of the transducer to induce ultrasonic vibrations in subcutaneous tissue.

Additionally or alternatively, in some such embodiments, the device is configured to allow application of suction to the skin surface apparent through the hole of the working face independently of activation of the transducer.

Configuration for simultaneous, alternating and/or independent activation of such functionalities is clear to a person having ordinary skill in the art and includes one or more of switches, wiring, power supplies and an appropriately-configured controller.

### Irradiation

In some embodiments, a device of the teachings herein that comprises a sonotrode having a hollow (whether or not having a conical portion) is configured to allow irradiation of a skin surface apparent through the hole of the working face of the sonotrode with electromagnetic radiation

As discussed in greater detail below, in some such embodiments, the device is functionally-associated with a radiation source comprising an aperture, which aperture is in optical communication with the hollow (in some embodiments through a waveguide). Activation of the radiation source leads to irradiation of a skin surface apparent through the hole of the working face of the sonotrode with electromagnetic radiation from the radiation source In some embodiments, the functionally-associated radiation source and/or optional waveguide are components of the device. Alternatively, in some embodiments, the functionally-associated radiation source and/or optional waveguide are not components of the device.

In some such embodiments, the device is configured to allow irradiation of the skin surface apparent through the hole of the working face simultaneously with activation of the transducer to induce ultrasonic vibrations in subcutaneous tissue.

Additionally or alternatively, in some such embodiments, the device is configured to allow irradiation of the skin surface apparent through the hole of the working face alternating with activation of the transducer to induce ultrasonic vibrations in subcutaneous tissue.

Additionally or alternatively, in some such embodiments, the device is configured to allow irradiation of the skin surface apparent through the hole of the working face independently of activation of the transducer.

Configuration for simultaneous, alternating and/or independent activation of such functionalities is clear to a person having ordinary skill in the art and includes one or more of switches, wiring, power supplies and an appropriately-configured controller.

In some such embodiments, the device is configured for at least three functions:
to allow irradiation of a skin surface apparent through the hole of the working face of the sonotrode with electromagnetic radiation;
to allow application of suction to the skin surface apparent through the hole of the working face; and
to induce ultrasonic vibrations in subcutaneous tissue on activation of the transducer.
   In some embodiments, such a device is configured to allow simultaneous activation of at least two functions selected from the group consisting of: the irradiation of a skin-surface; the application of suction; and activation of the transducer.

Additionally or alternatively, in some embodiments, such a device is configured to allow alternating activation of at least two functions selected from the group consisting of: the irradiation of a skin-surface; the application of suction; and activation of the transducer.

Additionally or alternatively, in some embodiments, such a device is configured to allow independent activation of at least two functions selected from the group consisting of: the irradiation of a skin-surface; the application of suction; and activation of the transducer.

Configuration for simultaneous, alternating and/or independent activation of such functions is clear to a person having ordinary skill in the art and includes one or more of switches, wiring, power supplies and an appropriately-configured controller.

In embodiments where a device is configured to irradiate a skin surface apparent through the hole of the working face of the sonotrode with electromagnetic radiation (whether or not having a conical portion), the irradiating is with electromagnetic radiation having a wavelength in any suitable range. In some embodiments, the range selected from the group consisting of:
UV light (having wavelengths in the range of 10 to 400 nm).
visible light (having wavelengths in the range of 400 to 750 nm);
IR light (having wavelengths in the range of 750 nm to 15 micrometers);
terahertz radiation (having wavelengths in the range of 10 micrometers to 1 mm (30 to 0.3 THz)); and
microwave radiation (having wavelengths in the range of 1 mm to 1 m (300 GHz to 0.3 GHz)).

In embodiments configured for irradiation with UV light, preferred UV light is UV-C (100-280 nm), UV-B (280-315 nm) and/or UV-A (315-400 nm).

In embodiments configured for irradiation with IR light, preferred IR light is NIR light (having wavelengths in the range of 750 nm to 1.4 micrometer); short IR light (having wavelengths in the range of 1.4 micrometer to 3 micrometer); midwave IR light (having wavelengths in the range of 3 micrometer to 8 micrometer); and longwave IR light (having wavelengths in the range of 8 micrometer to 15 micrometer).

In some such embodiments, the irradiating of a skin surface apparent through the hole of the working face with radiation is illuminating a skin surface apparent through the hole of the working face with light (i.e., IR light, visible light, UV light).

The wavelength of the electromagnetic radiation is typically selected as a wavelength that has a useful effect on bodily tissue such as light having a wavelength known in the art of transdermal subcutaneous tissue treatment, e.g., 1060 nm.

In some embodiments, the device is configured so that the radiation propagates in an axial direction from a proximal end of the hollow towards the hole of the working face which is the open end of the hollow. In some alternative embodiments, the device is configured so that the radiation enters the hollow in a non-axial direction from a location different from the proximal end of the hollow.

In some embodiments, the configuration of the device for such irradiation is that the device comprises a waveguide having a proximal end associable with the aperture of a radiation source (the part of a radiation source from which the radiation emerges) and a distal end of the waveguide leads to inside the hollow of the sonotrode, the waveguide providing optical communication from a radiation source to inside the hollow. As a result, radiation generated by a radiation source functionally associated with the proximal end of the waveguide is directed by the waveguide from the aperture of an associated radiation source into the hollow of the sonotrode. In such embodiments, any radiation source having any dimensions can be used as long as a suitable waveguide exists and can be a component of the device as described herein. In some such embodiments, the radiation source is a component of the device. Alternatively, in some such embodiments, the radiation source is not a component of the device. As discussed in greater detail hereinbelow, in some embodiments, a portion of the waveguide passes through components of the device (e.g., the transducer) in parallel to the sonotrode axis and, in some such embodiments, enters the hollow from the proximal end thereof. Alternatively, in some embodiments a portion of the waveguide passes through a non-axial through-channel that provides communication between the hollow and outside of the sonotrode through the wall which inner surface defines the hollow (in some embodiments being the conical wall). For light radiation, suitable waveguides include optical fibers and light pipes. For microwave and terahertz radiation, suitable waveguides include waveguides, for example, flexible small-dimension waveguides such as dielectric waveguides or waveguides available from Fairview Microwave, Inc. (Lewisville, TX, USA).

Alternatively, in some embodiments, the configuration of the device for such irradiation is that the device further comprises a radiation source and is devoid of a waveguide. In some such embodiments, the radiation source is located inside the hollow. In some such embodiments, the radiation source is located inside a physical component of the sonotrode. In some embodiments, the aperture of the source is directed into the hollow of the sonotrode from the proximal end of the hollow. Alternatively, in some embodiments, the aperture of the source is directed to inside the hollow of the sonotrode through a non-axial through-channel that provides communication between the hollow and outside of the sonotrode through the wall which inner surface defines the hollow (in some embodiments being the conical wall). In some embodiments, radiation from the aperture propagates in parallel with the sonotrode axis. In some embodiments, radiation from the aperture propagates not in parallel with the sonotrode axis.

### Radiation Sources

A radiation source, whether part of the device or not, is any suitable radiation source.

For light radiation (UV, visible, IR), any suitable source of light radiation may be used. In some such embodiments a suitable light source includes a laser such as a diode laser, solid-state laser or a semiconductor laser for producing light of a desired wavelength. In some embodiments, a suitable light source comprises a source of non-coherent light such as an LED, a flashlamp (e.g., a halogen lamp such as Xe or Kr) or other source of intense pulsed light (IPL).

For microwave radiation. any suitable source of microwave radiation may be used. In some such embodiments a suitable source comprises a magnetron, preferably a miniature magnetron (such as available from Sunchonglic, Guangdong, China) for generating microwave radiation of a desired wavelength.

For terahertz radiation. any suitable source of terahertz radiation may be used. In some such embodiments a suitable source comprises a terahertz source, preferably a miniature source (such as available from TeraSense Group Inc, San Jose, CA, USA) for generating terahertz radiation having a desired wavelength.

### Reflective Surface

In some embodiments, at least part of the inner surface of the hollow (e.g., the inner conical surface) is configured to be reflective (diffusely reflective and/or specularly reflective) to the radiation, in some embodiments at least 50%, at least 60%, at least 80% and even at least 90% of the inner surface of the hollow is reflective. In some embodiments by reflective is meant that reflectance of the reflective portion of the surface is at least 60% at normal incidence, more preferably at least 70%, at least 80%, at least 90% and even at least 95% reflectance at normal incidence. In such embodiments, radiation that contacts the inner surface of the hollow is reflected to potentially irradiate a skin-surface apparent through the hole of the working face. A person having ordinary skill in the art is familiar with materials suitable for making an inner surface of the hollow of a sonotrode reflective to a desired degree for radiation of a specified wavelength without undue experimentation. For example, in some embodiments when the radiation is light, the inner surface of the hollow is mirrored, for example, by polishing or coating the inner surface of an aluminum sonotrode, e.g., by silvering, plating, vapor deposition, e-beam deposition, ion-assisted e-beam deposition of a reflective metal layer such as silver and, if required, coating with a protective layer to prevent formation of a non-reflective oxide layer. In some embodiments, at least part of the part of the inner surface of the hollow that is configured to be reflective is a silver mirror. Additionally or alternatively, in some embodiments, at least part of the part of the inner surface of the hollow configured to be reflective is an aluminum mirror. That said, in preferred embodiments, the inner surface of the hollow is diffusely reflective.

### Optical Element

In embodiments where a device is configured to irradiate a skin surface apparent through the hole of the working face of the sonotrode with electromagnetic radiation (whether or not having a conical portion), the device further comprises at least one optical element to refract the radiation. Typically, an optical element is configured to refract the radiation in order to:
direct at least some of the radiation towards the open end of the hollow;
direct at least some of the radiation away from the inner surface of the hollow;
distribute the radiation in a desired manner at the open end of the hollow.
   For instance, in some embodiments, an optical element is configured to spread out a beam of radiation from the radiation source to be more evenly distributed over the area of the open end of the hollow, e.g., like a concave lens for light. For instance, in some embodiments, an optical element is configured to change the direction of a beam of radiation from being directed towards the inner surface of the hollow to be directed towards the open end of the hollow. In some such embodiments, the optical element is inside the hollow of the sonotrode and/or inside a physical component of the sonotrode. For light radiation, suitable optical elements include lenses, prisms and diffraction gratings. For microwave radiation, suitable optical elements include lens antennae such as delay lens, fast lens, dielectric lens, constrained lens, Fresnel zone lens and Luneburg lens. For terahertz radiation, suitable optical elements include terahertz lenses such as available from Menlo Systems GmbH. Planegg, Germany

### Pulsed Ultrasonic Treatment

According to an aspect of some embodiments of the teachings herein, there is also provided a device for treatment of tissue with ultrasonic vibrations, the device comprising:
i. a sonotrode with a working face;
ii. functionally associated with the sonotrode, an ultrasonic transducer,
iii. functionally associated with the ultrasonic transducer, an ultrasound power supply configured to provide an alternating current (AC) oscillating at an ultrasonic driving frequency to drive the ultrasonic transducer, and
iv. a controller configured to receive a user-command to cause the working face to vibrate at an ultrasonic frequency and, subsequent to receipt of such a command, to activate other components of the device to cause the working face to periodically ultrasonically vibrate at a rate of at least 2 pulses per second, each pulse having a duration of less than 250 millisecond and any two pulses separated by a rest phase of at least 10 milliseconds.

According to an aspect of some embodiments of the teachings herein, there is also provided a method for treatment of tissue with ultrasonic vibrations, the method comprising:
acoustically coupling working face of a sonotrode with a tissue surface;
for a treatment duration, causing the working face to periodically vibrate at an ultrasonic frequency at a rate of at least 2 pulses (of ultrasonic vibrations) per second,
each pulse having a duration of less than 250 millisecond and any two pulses separated by a rest phase of at least 10 milliseconds,
   wherein the intensity of the pulses and the treatment duration are sufficient to achieve a desired result.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIG. 1 (prior art) schematically depicts a device for application of ultrasonic vibrations into a medium through a surface of the medium;

FIGS. 2A, 2B, 2C and 2D (prior art) schematically depict different sonotrodes configured to function as acoustic amplitude transformers: FIG. 2A linear taper sonotrode;

FIG. 2B exponential taper sonotrode; FIG. 2C stepped taper sonotrode; and FIG. 2D mushroom sonotrode according to US 2011/0213279;

FIG. 3 (prior art) schematically depicts an embodiment of a sonotrode according to US 2019/0091490;

FIGS. 4A, 4B, 4C and 4D schematically depict a device and a sonotrode according to an embodiment of the teachings herein configured for application of suction to a skin surface: FIG. 4A the device in side view, FIG. 4B the sonotrode in side view, FIG. 4C the sonotrode in side cross section, and FIG. 4D the sonotrode in perspective in a view from the bottom towards the working face;

FIG. 5 schematically depicts an embodiment of a sonotrode according to an embodiment of the teachings herein configured for irradiating skin with radiation, specifically illuminating skin with light;

FIG. 6 schematically depicts an embodiment of a sonotrode according to an embodiment of the teachings herein;

FIG. 7 schematically depicts an embodiment of a sonotrode according to the teachings; herein configured for application of suction to a skin surface

FIGS. 8A and 8B schematically depict an embodiment of a device according to the teachings herein configured for both irradiating skin with radiation, specifically and illuminating skin with light and for application of suction to a skin surface: FIG. 8A is the device in side view and FIG. 8B is the sontrode of the device in side cross section;

FIGS. 9A and 9B each schematically depicts embodiments of a device according to the teachings herein configured for irradiating skin with radiation in side cross section; and

FIGS. 10A and 10B each schematically depicts an embodiment of device suitable for treatment of tissue with pulses of ultrasonic vibrations.

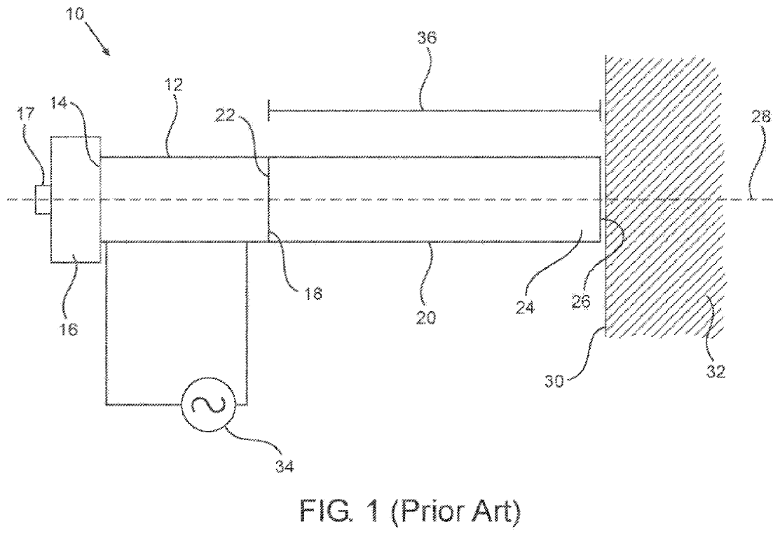

### DESCRIPTION OF SOME EMBODIMENTS OF THE INVENTION

The invention, in some embodiments, relates to the treatment of body tissue with energy and more particularly, but not exclusively, to devices for treatment of subcutaneous fat by transdermally-inducing ultrasonic vibrations in subcutaneous tissue and/or transdermally delivering energy with electromagnetic radiation such as light to subcutaneous tissue. In some embodiments, the treatment of the subcutaneous tissue is effective in reducing the amount of subcutaneous fat therein. In some embodiments, transdermal radiation-delivery of energy and transdermal induction of ultrasonic vibrations in subcutaneous tissue can be performed simultaneously, alternatingly or in an unrelated (independent) fashion. In some embodiments, the device simultaneously transdermally induces both ultrasonic transverse and ultrasonic longitudinal vibrations in subcutaneous tissue.

The principles, uses and implementations of the teachings herein may be better understood with reference to the accompanying description and figures. Upon perusal of the description and figures present herein, one skilled in the art is able to implement the invention without undue effort or experimentation. In the figures, like reference numerals refer to like parts throughout.

Before explaining at least one embodiment in detail, it is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

As discussed above, in patent publication US 2019/0091490 some of the Inventors disclosed a sonotrode found to be particularly effective in treating subcutaneous tissue. The Inventors believe that the efficacy of that sonotrode is at least partially due to the sonotrode simultaneously inducing both ultrasonic transverse and ultrasonic longitudinal vibrations in subcutaneous tissue to acoustically deliver energy to treat the tissue.

Until recently, the Inventors believed that simultaneous induction of both ultrasonic transverse vibrations and ultrasonic longitudinal vibrations, both with sufficient intensity to deliver substantial energy where the two modes are balanced to achieve a desired biological effect, is only possible with a sonotrode configured according to the teachings of US 2019/0091490.

Herein are disclosed devices for treatment of subcutaneous tissue and methods of using the devices that include a sonotrode having a conical portion and a ring-shaped working face. It has been surprisingly found that a device according to such embodiments of the teachings herein is particularly effective in treating subcutaneous tissue. Without wishing to be held to any one theory, it is currently believed that the efficacy is at least partially due to the sonotrode simultaneously inducing both ultrasonic transverse and ultrasonic longitudinal vibrations in subcutaneous tissue to acoustically deliver energy to treat the subcutaneous tissue. It is currently believed that both induced modes of vibrations have sufficient intensity to deliver substantial energy to achieve a desired biological effect, e.g., substantial heating of tissue and substantial disrupting of adipocytes in a manner that rivals and even exceeds the device disclosed in US 2019/0091490 despite the now-disclosed sontrode being entirely different from the sonotrode of US 2019/0091490.

A challenge in operating a device according to the teachings of US 2019/0091490 is that the longitudinal waves generated by the ultrasonic transducer raise the temperature of the central portion of the working face of the sonotrode. The temperature of the central portion may rise to a degree that can cause discomfort or even damage to a treated subject. As a result, an operator of such a device must limit the power of the ultrasonic vibrations generated by the transducer to reduce the degree of working face heating and also take special care when using the device to avoid discomfort or damage to the treated subject. In contrast, the ring-shaped working face of a sonotrode of a device of the teachings herein does not suffer from such heating as the ring-shaped working face has no central portion, only a hole. Some embodiments of the devices and sonotrodes disclosed herein have additional advantages as disclosed hereinbelow.

According to an aspect of some embodiments of the invention there is provided a device suitable for treating subcutaneous tissue, comprising:
a. an ultrasonic transducer for generation of ultrasonic vibrations having a proximal face and a distal face; and
b. a sonotrode with a sonotrode axis including:
   i. a proximal face in contact with and acoustically-coupled to the distal face of the ultrasonic transducer,
   ii. a conical portion having a smaller-radius proximal end and a larger-radius distal end, wherein the conical portion is defined by a conical wall having an outer conical surface and an inner conical surface, which inner conical surface at least partially defines a hollow, and
   iii. a ring portion extending radially outwards from the distal end of the conical portion having a ring-shaped proximal face and a ring-shaped distal face, said ring-shaped distal face being the working face of the sonotrode, the hole of the working face constituting an open end of the hollow.

In the summary section, this aspect and additional aspects of the teachings herein are described, two of the additional aspects relating to a device comprising an ultrasonic transducer and a sonotrode having an open-ended hollow and a device comprising a transducer with a hollow axial bolt. As is clear to a person having ordinary skill in the art, the detailed description herein and figures describe the components and operation of this aspect. of the teachings herein

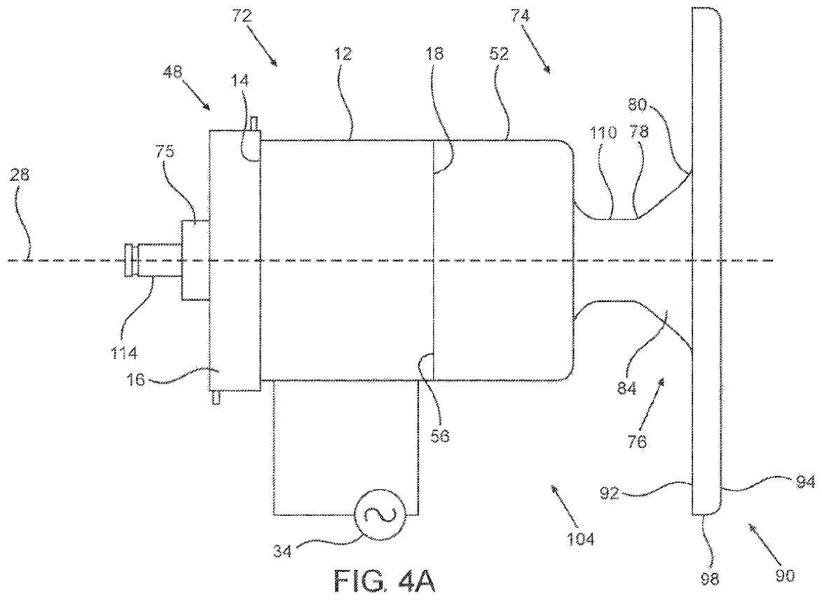

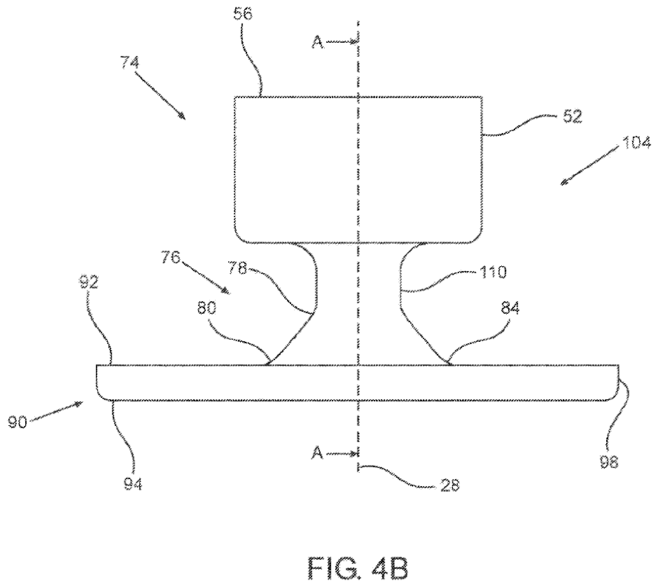

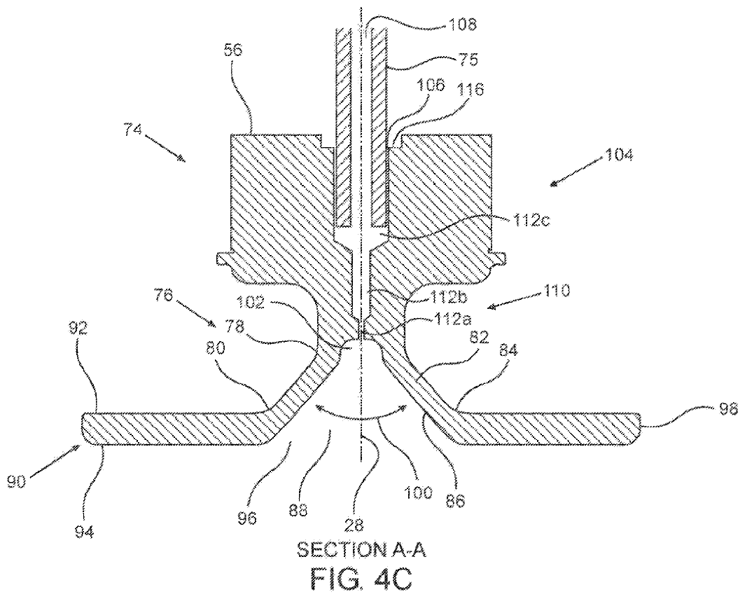

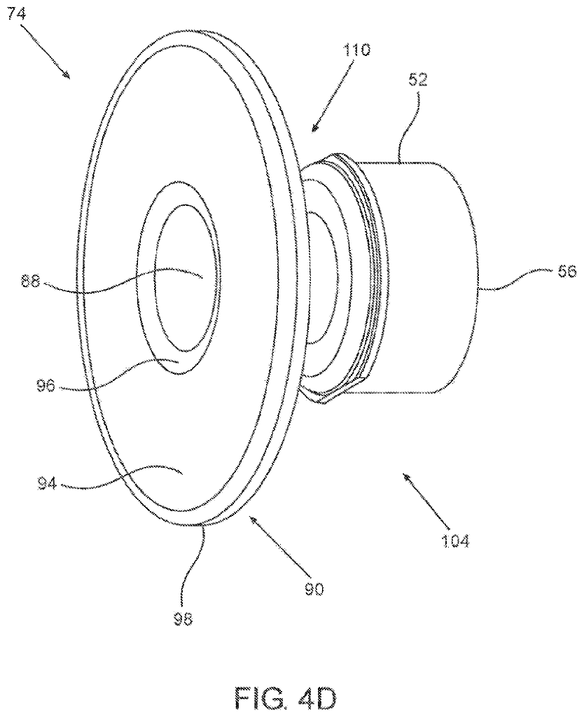

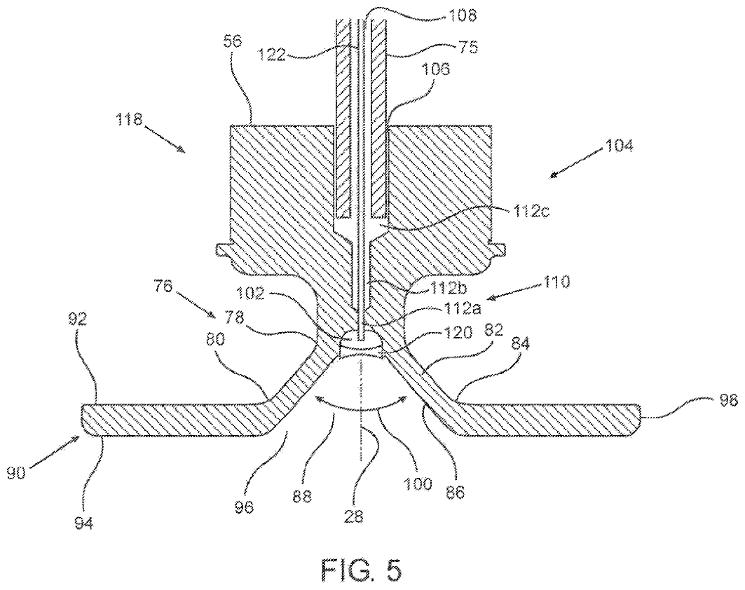

A representative embodiment of the device according to the teachings herein, a device 72, is schematically depicted in FIGS. 4A-4D: FIG. 4A (device 72 in side view with an ultrasonic transducer 12 and a sonotrode 74), FIG. 4B (sonotrode 74 in side view), FIG. 4C (sonotrode 74 in side cross section view) and FIG. 4D (sonotrode 74 in a perspective view from the bottom). Device 72 is configured for transdermally-inducing ultrasonic vibrations in subcutaneous tissue through the working face of sonotrode 74 when transducer 12 is activated together with the simultaneous, alternating or independent application of suction through the hole in the working face as is discussed in greater detail hereinbelow.

Ultrasonic transducer 12 has a proximal face 14 and a distal face 18. Ultrasonic transducer 12 is a Langevin-type prestressed (at between 45 N/m to 100 N/m) transducer that includes a stack of four 6 mm diameter disks, configured to produce ultrasonic longitudinal frequencies of between 56 kHz to 60 kHz, held together with an acoustic reflector 16 and with sontrode 74 by an axial bolt 75.

Sonotrode 74 has sonotrode axis 28 and includes:
i. a proximal face 56 in contact with and acoustically-coupled to distal face 18 of ultrasonic transducer 12,
ii. a conical portion 76 having a smaller-radius proximal end 78 and a larger-radius distal end 80, wherein conical portion 76 is defined by a conical wall 82 having an outer conical surface 84 and an inner conical surface 86, which inner conical surface 86 at least partially defines a hollow 88, and
iii. a ring portion 90 extending radially outwards from a distal end 80 of the conical portion 76 having a ring-shaped proximal face 92 and a ring-shaped distal face which is the working face 94 of sonotrode 74 and of device 72, the hole 96 of working face 94 constituting an open end of hollow 88.

### Sonotrode Material

Sonotrode 74 is a monolithic block of aluminum 6061 (an alloy of aluminum that includes magnesium and silicon as alloying elements) so that all the components are integrally formed. Working face 94 of sonotrode 74 includes a 10 micrometer thick soft anodization layer.

### Ring Portion

The ring portion has a ring-shaped proximal face (92 in FIG. 4), a ring-shaped distal face which is the working face of the sonotrode (94 in FIG. 4) and a peripheral wall (98 in FIG. 4).

In preferred embodiments, the shape of a ring portion of a sonotrode is a circle (when viewed in parallel to the sonotrode axis), preferably centered around the sonotrode axis. The outer periphery of ring portion 90 of sonotrode 74 is a circle when viewed in parallel to sonotrode axis 28. In some alternate embodiments, the ring portion has a different shape such as an oval or ellipse.

In preferred embodiments, the diameter of the ring portion (the greatest dimension of the ring portion that is perpendicular to the sonotrode axis) is between 20 mm and 300 mm (and in some embodiments up to 200 mm) and is typically selected, inter alia, based on the intended use (what portion of the body is to be treated, arms preferably treated with a smaller diameter and thighs preferably treated with a greater diameter ring portion) and on a selected driving frequency as discussed below. Ring portion 90 of sonotrode 74 has a diameter of 90 mm.

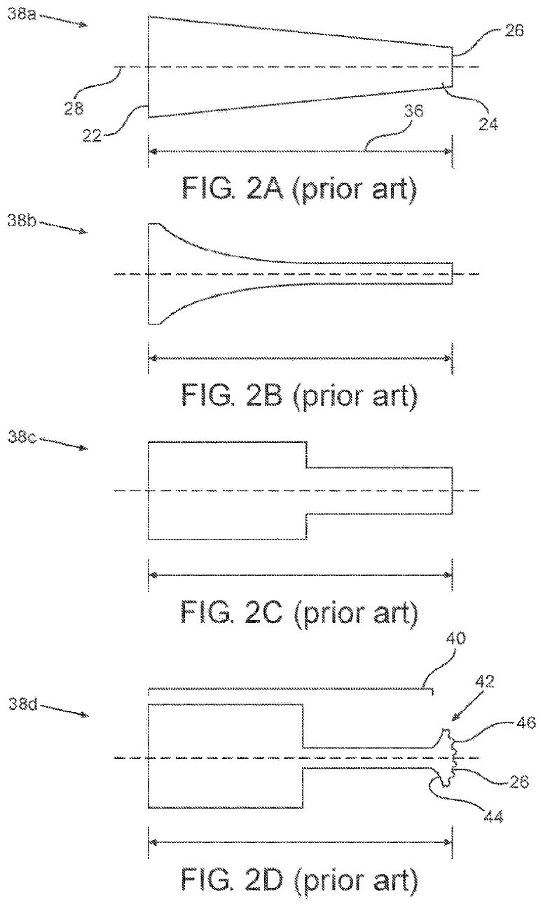

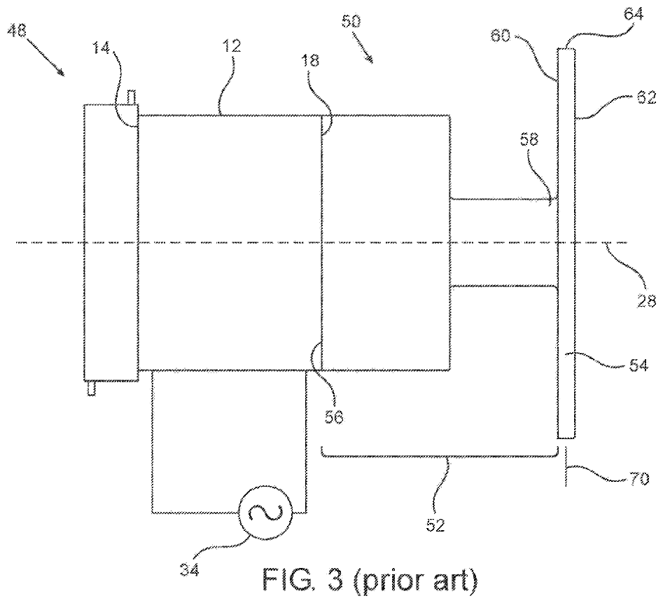

In preferred embodiments, at least 80% and even at least 90% of the surface area of the working face is perpendicular to the sonotrode axis. In FIGS. 2, more than 90% of working face 94 of sonotrode 74 is perpendicular to sonotrode axis 28 with only a small peripheral portion near the intersection with peripheral wall 98 curving upwards in a proximal direction to avoid scratching, wounding or causing discomfort to a person being treated. In some alternate embodiments, less than 90% of the working face is perpendicular to the sonotrode axis. In some such alternate embodiments, a portion of the working face (at least 20%, at least 30%, at least 50% and even at least 70%) is convexly curved in a proximal direction so that, in cross section parallel to the sonotrode axis, the ring portion has a convex lenticular shape. In some such alternate embodiments, a portion of the working face (at least 20%, at least 30%, at least 50% and even at least 70%) is flat but not parallel to the sonotrode axis so that, in cross section (when viewed perpendicular to the sonotrode axis) the portion of the working face is a straight line.

In preferred embodiments, at least 90% of the surface area proximal face is perpendicular to the sonotrode axis. 100% of proximal face 92 of sonotrode 74 is perpendicular to sonotrode axis 28. In some alternate embodiments, less than 90% of the proximal face is perpendicular to the sonotrode axis. In some such alternate embodiments, a portion (at least 20%, at least 30%, at least 50% and even at least 70%) is convexly curved in a distal direction so that, in cross section perpendicular to the sonotrode axis, the ring portion has a lenticular shape. In some such alternate embodiments, a portion of the proximal face (at least 20%, at least 30%, at least 50% and even at least 70%) is flat but not parallel to the sonotrode axis so that, in cross section (when viewed perpendicular to the sonotrode axis) the portion of the proximal face is a straight line.

In some embodiments, the intersection of the working face with the peripheral wall is not curved. Alternately, in some preferred embodiments the intersection of the working face with the peripheral wall is curved reducing the chance of scraping or scratching a skin surface during use. In sonotrode 74, the intersection of working face 94 and peripheral wall 98 is curved.

In some embodiments, the intersection of the proximal face with the peripheral wall is not curved. Alternately, in some preferred embodiments the intersection of the proximal face with the peripheral wall is curved. In sonotrode 74, the intersection of proximal face 92 and peripheral wall 98 is not curved, being 90°.

In some embodiments, at least some of the peripheral wall is parallel to the sonotrode axis, preferably at least 20%, at least 30%, at least 40% and even at least 50%. of the peripheral wall is parallel to the sonotrode axis. In sonotrode 74, 60% of peripheral wall 98 is parallel to sonotrode axis 28. In some embodiments, the central portion of the peripheral wall is parallel to the sonotrode axis. In sonotrode 74, the central portion of peripheral wall 98 is parallel to sonotrode axis 28. In some alternate embodiments, the central portion of the peripheral wall is not parallel to the sonotrode axis. In some such alternate embodiments, the central portion of the peripheral wall is curved (e.g., the entire peripheral wall is curved). In alternate such alternate embodiments, the central portion of the peripheral wall is straight and not parallel to the sonotrode axis so that either the diameter of the proximal face is greater than the diameter of the distal face, or the diameter of the distal face is greater than the diameter of the proximal face.

In some preferred embodiments, at least 70%, at least 80% and even at least 90% of the surface areas of the working face and the proximal face are parallel (and preferably perpendicular to the sonotrode axis). In such embodiments, the thickness of the working face (the dimension parallel to the sonotrode axis) as measured at a parallel portion is any suitable thickness, preferably at least 1 mm and not more than 10 mm. In some embodiments, to increase the robustness of the ring portion, the thickness is at least 2 mm and even at least 3 mm. In some embodiments, the thickness is not more than 8 mm and even not more than 7 mm. In sonotrode 74, at least 90% of the surfaces of working face 94 and proximal face 92 are parallel, and the ring portion is 5 mm thick. In some alternate embodiments, less than 70% of the surface areas of the working face and the proximal face are parallel, e.g., when one or both faces are curved and/or one or more of the faces are flat but not parallel. In such alternate embodiments, the thickness of the ring portion at the thickest portion and at the thinnest portion is preferably at least 1 mm and not more than 20 mm (and in some embodiments not more than 10 mm) where the difference between the thickness of the thickest portion and the thickness of the thinnest portion is not more than 7 mm, not more than 5 mm, not more than 3 mm, not more than 2 mm and even not more than 1 mm

### Hole in Working Face

The working face is ring-shaped, having a hole which constitutes the open end of the hollow. In some instances when the sonotrode is used, the working face contacts a ring-shaped portion of the skin surface, allowing induction of vibrations in subcutaneous tissue in the usual way. A different portion of the skin surface that is encircled by the ring-shaped portion of the skin surface is apparent in the hole in the working face of the sonotrode, the different portion of the skin closing the hollow from fluid communication with the open air.

In preferred embodiments, the shape of the hole is a circle (when viewed in parallel to the sonotrode axis), preferably centered around the sonotrode axis. The shape of hole 96 of ring portion 90 of sonotrode 74 is a circle when viewed in parallel to sonotrode axis 28. Hole 96 of sonotrode 74 is centered around sonotrode axis 28. In some alternate embodiments, the hole has a different shape such as an oval or ellipse and/or is not centered around the sonotrode axis.

In preferred embodiments, the diameter of the hole (the greatest dimension of the hole that is perpendicular to the sonotrode axis) is between 10% and 70% of the diameter of the ring portion, more preferably between 20% and 50% and even more preferably between 25% and 40%. Hole 96 of sonotrode 74 is a circle having a 30 mm diameter, so is 33% of the 90 mm diameter of ring portion 90.

### Conical Surfaces and Hollow

A sonotrode according to the teachings herein has a conical portion having a smaller-radius proximal end and a larger-radius distal end, wherein the conical portion is defined by a conical wall having an outer conical surface and an inner conical surface, which inner conical surface at least partially defines a hollow. As is clear from this description, the conical portion is a hollow conical portion, i.e., has a hollow, the hollow at least partially defined by. the inner conical surface

In preferred embodiments, the outer conical surface and the inner conical surface are parallel so that the thickness of the conical wall is constant. In such embodiments, the thickness of the conical wall is any suitable thickness, typically between 2 mm and 10 mm, in some preferred embodiments between 2 mm and 6 mm. In sontrode 74, outer conical surface 84 and inner surface 86 are parallel, conical wall 82 having a constant thickness of 3.3 mm. In some alternate embodiments, the outer surface and inner surface are not parallel and the thickness of the conical wall is not constant. In preferred such alternate embodiments, the thickness of the conical wall varies within the range of 2 mm and 10 mm, preferably more proximal portions being thicker than more distal portions.

The conical angle of inner surface is any suitable angle. In preferred embodiments, when the shape of the hole is a circle and the inner surface defines a portion of a right circular cone, there is a single conical angle, preferably between 70° and 95°, more preferably between 75° and 90° and even more preferably between 78° and 86°. In sontrode 74, hole 96 is a circle and inner surface 86 defines a right circular cone so there is a single conical angle 100 of 82°. In some alternate embodiments, e.g., when the shape of the hole is not a circle, e.g., an oval or ellipse, or the inner surface defines a portion of an oblique cone there are a multiplicity of conical angles from a smallest to a greatest conical angle. In preferred such alternate embodiments, both the smallest and the greatest conical angles are between 70° and 95°. In preferred embodiments, the inner surface defines a portion of a right cone where a line between the (imaginary) apex of the cone and the center of the hole is perpendicular to the plane of the hole (whether or not the hole is a circle). In some embodiments, the inner surface defines a portion of a cone that is not a right cone: in such embodiments the angle between a line between the (imaginary) apex of the cone and the center of the hole is close to perpendicular (90°) preferably being not less than 70°, not less than 75°, not less than 80° and even not less than 85°.

In some embodiments, the conical inner surface extends to the working face and defines the hole of the sonotrode. In sonotrode 74, conical inner surface 86 extends to working face 94, thereby defining hole 96. In some alternate embodiments, the distal portion of the inner surface is not conical. In some such embodiments, the distal portion of the inner surface that defines the inner part of the ring portion is parallel to the sonotrode axis.

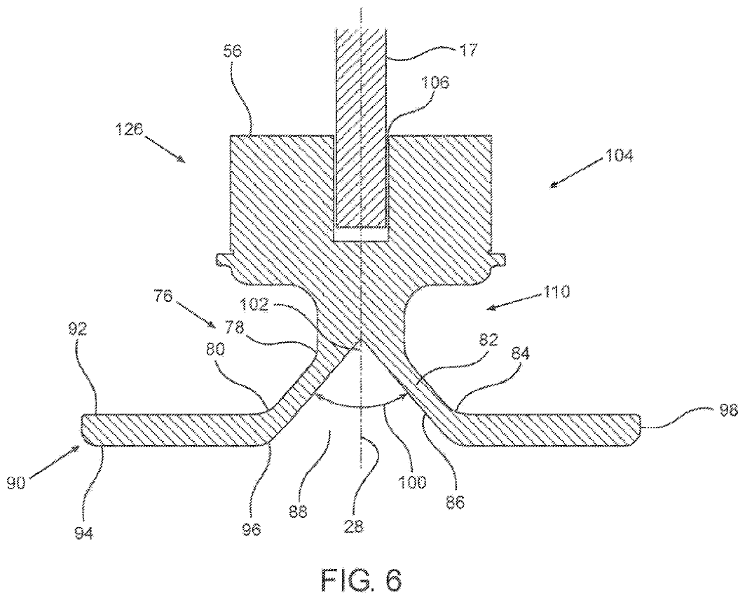

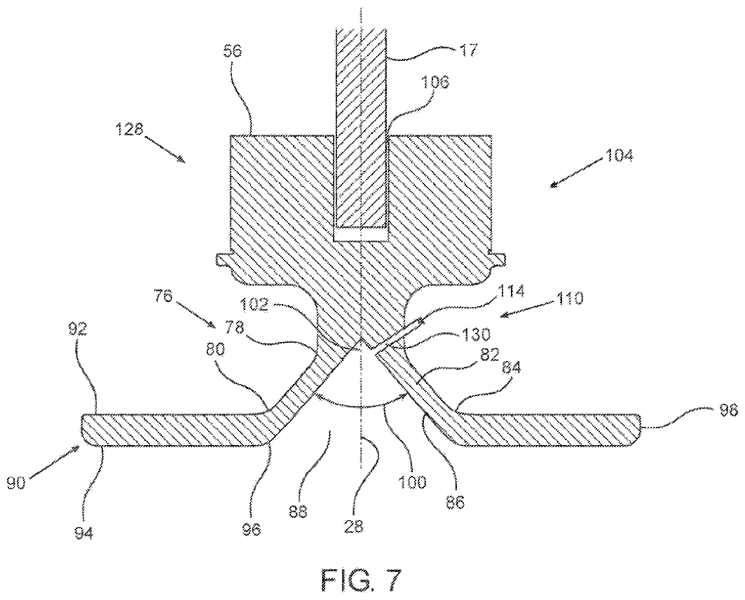

In some embodiments, the inner conical surface is a complete cone, ending at a pointed or curved apex. In such embodiments, the portion of the hollow defined by the inner conical surface is a true cone (see FIGS. 6 and 7). In alternate embodiments, the inner conical surface and the portion of the hollow defined by the inner conical surface are truncated cones. In sontrode 74, conical inner surface 86 and the portion of hollow 88 defined by conical inner surface 86 is a truncated right circular cone. The height (dimension parallel to sonotrode axis) of the portion of the hollow that is defined by the conical inner surface is any suitable height and is defined by the dimensions of other features of the sonotrode. In sonotrode 74, the height of the portion of hollow 88 that is defined by conical inner surface 86 is 12 mm.

In some embodiments where the inner conical surface and the portion of the hollow defined by the inner conical surface are truncated cones, there is a proximal hollow wall that is perpendicular to the working face so that at least a portion of the hollow is a true truncated cone. Alternatively, in some embodiments, the portion of the hollow that is above the proximal end of the inner conical surface is any suitable shape. In sonotrode 74, the portion of hollow 88 that is above the proximal end of inner conical surface 86 is a proximal portion 102. Proximal portion 102 of hollow 88 is an approximately cylindrical volume with curved edges having a 7 mm diameter (perpendicular to sonotrode axis 28) and a 5 mm height (dimension parallel to sonotrode axis 28).

### Stem

As noted above, a sonotrode has a proximal face that is in contact with, and acoustically-coupled to, the distal face of the ultrasonic transducer.

In some embodiments, the proximal end of the conical wall defines the proximal face of the sonotrode.

In preferred embodiments, the sonotrode comprises a stem, the stem having a proximal face that is the proximal face of the sonotrode and a distal end which is the proximal end of the conical wall. Sonotrode 74 comprises a stem 104 that includes a proximal face 56 of sonotrode 74 and a distal end which is proximal end 78 of conical wall 82. As known in the art of sonotrodes, in cross section (perpendicular to the sonotrode axis), the stem is preferably circular, although in some embodiments in cross section the stem has a different shape, e.g., ellipse or oval.

Typically, the stem has one or more features that allow acoustic-coupling of the sonotrode to the transducer. In sonotrode 74, stem 104 includes a 10 mm diameter threaded bore 106, configured to mate with axial bolt 75. When device 72 is assembled in the usual way of Langevin-type transducers, reflector 16, the components of transducer 12 and sonotrode 74 are threaded onto bolt 75. Bolt 75 is tightly screwed into threaded bore 106 (e.g., with a torque of 45-100 N/m) to compress the components together to ensure contact and acoustic coupling thereof as is known in the art of Langevin-type transducers.

In the art, axial bolts of Langevin-type transducers are regular solid bolts that have the mechanical properties required to compress the transducer components together under conditions of ultrasonic vibrations and concomitant heating. In some embodiments of the teachings herein, the axial bolt includes an axial passage (e.g., fluid communication such as of air, passage of a physical component and/or optical communication of light) between the proximal end and the distal end of the bolt. In preferred embodiments, the axial passage is colinear with the sontrode axis. Alternately, in some embodiments the axial passage is parallel with but not colinear with the sonotrode axis. Alternately, in some embodiments, the axial passage is not parallel with the sonotrode axis. The utility of such an axial passage is discussed hereinbelow. In sonotrode 74, axial bolt 75 includes an axial passage 108 that is colinear with sonotrode axis 28. In some embodiments, the axial bolt includes more than one axial passage, e.g., two, three or even more axial passages, typically that do not have fluid communication one with the other, for example two, three or even more axial passages, in some embodiments all parallel with the sonotrode axis.

In embodiments comprising a stem, the stem can have any suitable shape. In preferred embodiments, the sonotrode and the stem are together configured to function as an acoustic amplitude transformer for a selected ultrasonic frequency. In such embodiments, any configuration of the stem and sonotrode as known in the art for configuring the sonotrode to function as an acoustic amplitude transformer for the selected ultrasonic frequency can be used, such as by having a tapered stem as is discussed in the introduction with reference to FIGS. 2A-D.

Sonotrode 74 is configured to function as an acoustic amplitude transformer for a selected ultrasonic frequency by configuring stem 104 as a step-tapered stem (see FIGS. 2C and 2D). Specifically, stem 104 of sonotrode 74 includes a wide-diameter proximal stem portion 52 having a diameter of 42 mm which is the diameter of distal face 18 of transducer 12. Proximal stem portion 52 bears proximal face 56 (also called “input surface”) of sonotrode 74. Stem 104 further includes a narrow-diameter distal stem portion 110 having a 14 mm diameter. The transition from proximal stem portion 52 to distal stem portion 110 is not abrupt, rather edges and transitions are rounded for increased mechanical strength and avoiding sharp edges that can hurt or wound an operator.

The length (dimension in the axial direction) of sonotrode 72 is 50 mm. The length of proximal stem portion 52 is 24 mm which is 48% of the length of sonotrode 72. The length of distal stem portion 110 (from the distal end of proximal stem portion 52 to the proximal end 78b of conical wall 82) is 13.2 mm. As is known to a person having ordinary skill in the art, for stepped tapered stems of sonotrodes, it is advantageous that the wide-diameter proximal stem portion be between 45% and 55% of the length of the sonotrode, preferably between 46% and 54% and even more preferably between 47% and 53% of the length of the sonotrode.

### Use of Sonotrode for Treating Subcutaneous Tissue

As is known in the art and discussed in the introduction, for use of a sonotrode of the teachings herein for treating subcutaneous tissue, the working face is acoustically coupled with a skin surface (e.g., by direct contact with the skin or by indirect contact through a coupling substance, e.g., a liquid or gel). An alternating current oscillating at an ultrasonic driving frequency is supplied from an ultrasound power supply (e.g., power supply 34 in FIG. 4A) to drive the ultrasonic transducer. The transducer generates ultrasonic longitudinal vibrations with a frequency of the driving frequency. The generated longitudinal vibrations propagate through the sonotrode to the working face. Without wishing to be held to any one theory, the generated longitudinal vibrations pass through the stem and through the conical wall which passage causes the working face to vibrate both with longitudinal vibrations and some type of transverse vibrations (e.g., shear waves, Lamb waves). The ultrasonic vibrations of the working face transdermally induce both ultrasonic longitudinal vibrations and transverse vibrations in the subcutaneous tissue, thereby treating the tissue.

The driving frequency is any suitable ultrasonic frequency, preferably between 30 kHz to 200 kHz, more preferably between 40 kHz to 100 kHz, and even more preferably between 40 kHz and 80 kHz. However, when a given sonotrode is driven by an arbitrary driving frequency the transdermal induction of vibrations in the subcutaneous may be less efficient so that treatment of a subject may take longer, be less comfortable and/or be less effective.

In some preferred embodiments, the sonotrode is configured to operate at at least one selected ultrasonic driving frequency and the ultrasonic transducer is configured to generate the selected driving frequency when driven by a driving current alternating at the selected driving frequency.

In some embodiments, configuration to operate at a selected ultrasonic driving frequency is that the sonotrode is configured to function as an acoustic amplitude transformer for a selected ultrasonic frequency, for example, by including a tapered stem, as described above.

Alternatively or preferably additionally, in some embodiments, configuration to operate at a selected ultrasonic driving frequency is that the length of the sonotrode from the proximal face (56) to the working face (94) is:

nλ_{longitudinal}/2

where n is a positive integer greater than 0; and
λ_{longitudinal} is the wavelength of ultrasonic longitudinal waves in the sonotrode, which is primarily determined by the material from which the sonotrode is made. The length of sonotrode 74 is 50 mm. In some embodiments, the length of the sonotrode is set based on the longitudinal speed of sound through the sonotrode at room temperature (25° C.). In some alternate embodiments, the length of the sonotrode is set based on the longitudinal speed of sound through the sonotrode to an expected operating temperature (e.g., 36°-40° C.).

Alternatively or preferably additionally, configuration to operate at a selected ultrasonic driving frequency is that the diameter of the ring portion (90) is:

nλₜᵣₐₙₛᵥₑᵣₛₑ/2

where n is a positive integer greater than 0; and
λₜᵣₐₙₛᵥₑᵣₛₑ is the wavelength of ultrasonic transverse waves in the sonotrode, which is primarily determined by the material from which the sonotrode is made. The diameter of ring portion 90 of sonotrode 74 is 90 mm. In some embodiments, the diameter of the ring portion is set based on the transverse speed of sound through the sonotrode at room temperature (25° C.). In some alternate embodiments, the diameter of the ring portion is set based on the transverse speed of sound through the sonotrode to an expected operating temperature (e.g., 36°-40° C.).

Typically, a person designing a specific sonotrode according to the teachings herein first decides on approximate desired sonotrode dimensions that can practically and conveniently be handled by an operator and that are also suitable for treating a specific part of the body (e.g., abdomen, thighs, face, under the chin) and the material from which the sonotrode is to be made. In preferred embodiments, the leng°th of a sonotrode is between 20 mm and 200 mm and the diameter of the ring portion is between 20 mm and 200 mm. The designer then selects a desired selected driving frequency based, for example, on regulatory requirements, cost, or power supply/transducer availability. Once a selected driving frequency is chosen, the designer can identify the exact sonotrode length and ring portion diameter that is close to the approximate desired sonotrode dimensions.

### Proximal Channel

In some embodiments, a sontrode according to the teachings herein further comprises a proximal channel between the hollow and outside of the sonotrode near the proximal face of the sonotrode and, in preferred embodiments, between the hollow and the proximal face of the sonotrode. In some embodiments, the proximal channel provides fluid communication (e.g., of air or other fluid) between the hollow and the outside near the proximal end of the sonotrode. Alternatively or additionally, in some embodiments, the proximal channel provides for the passage of a physical component (e.g., a waveguide such as a light guide such as an optical fiber) between the hollow and the outside. As discussed in detail hereinbelow, in some embodiments the proximal channel is configured to connect to a suction generator such as a vacuum pump, allowing evacuation of air from the hollow during operation of the device by application of suction through the proximal channel. In some embodiments, the proximal channel is configured to allow passage of a waveguide such as a light guide such as an optical fiber, allowing illumination with light of a skin-surface apparent through the hole of the working face of the sonotrode from inside the hollow.

As seen in FIG. 4C, sonotrode 74 includes a 3-portion proximal channel, collectively numbered 112, coaxial with axis 28 and providing fluid communication between proximal portion 102 of hollow 88 and proximal face 56 of sonotrode 74. Along the entire length, proximal channel 112 has a circular cross section and includes:
a 1 mm diameter by 3.1 mm long distal portion 112a,
a 3 mm diameter by 11.1 mm long middle portion 112b, and
a 1.8 mm long conical proximal portion 112c that widens from 3 mm diameter at the transition from middle portion 112b to 10 mm at the transition to threaded bore 106.

### Proximal Channel for Application of Suction

In some embodiments, the device is configured to apply suction to a skin-surface through the hole of the working face of the sonotrode by evacuation of air from the hollow during operation of the device. In some such embodiments that include a proximal channel, the proximal channel is configured to be connected to a suction generator such as a vacuum pump and the proximal channel allows evacuation of air from the hollow during operation of the device by activation of the suction generator.

Device 72 depicted in FIG. 4 is configured to apply suction to a skin-surface through the hole of the working face during operation by including a connector 114 (see FIG. 4A) which allows connecting proximal channel 112 to a suction generator such as a vacuum pump via axial passage 108 of axial bolt 75. Device 72 is further configured to apply suction to a skin surface by having a cylindrical 14 mm diameter/2 mm deep hole 116 coaxial with axis 28 in proximal face 56 of sonotrode 74. When transducer 12 and sonotrode 74 are held together by axial bolt 75, an appropriately-sized silicone rubber O-ring (not depicted) is seated inside hole 116 is compressed inside the walls of hole 116, the outer surface of axial bolt 75 and distal face 18 of transducer 12, making an air-tight seal that prevents air from leaking in from the transducer/sonotrode interface. Hole 116 can optionally be considered to be the most proximal section of proximal channel 112.

For use, device 72 is prepared in the usual way known in the art of sonotrodes, including by functionally-associating transducer 12 with a power supply 34 and connecting connector 114 to a suction generator (not depicted) such as a Venturi pump. A lubricant such as mineral oil is applied to the area of skin that is to be treated. Power supply 34 and the suction generator are activated and working face 94 is contacted with the surface of skin that is to be treated, with continuous back-and-forth or circular motion as is known in the art of transdermal subcutaneous tissue treatment. The suction generator draws air through connector 114, axial passage 108 in bolt 75, proximal channel portion 112c, middle channel portion 112b, distal channel portion 112a and from hollow 88, generating a low pressure in hollow 88, typically so that the pressure in hollow 88 is below 525 mm Hg (70 kPa) and preferably below 450 mm Hg (60 kPa) but above 100 mm Hg (13.4 kPa) and even above 200 mm Hg (27 kPa). In some preferred embodiments, the pressure in hollow 88 is between 200 mm Hg (27 kPa) and 300 mm Hg (40 kPa). In some alternate preferred embodiments, the pressure in hollow 88 is between 250 mm Hg (33 kPa) and 350 mm Hg (47 kPa), e.g., about 300 mm Hg (40 kPa). As a result of the low pressure in hollow 88, working face 94 makes better contact with the skin to be treated, thereby more efficiently and consistently inducing ultrasonic vibrations in subcutaneous tissue. Further, the suction applied to the portion of skin located in hole 96 while sonotrode 74 is moved has a pleasant massaging effect that increases a subject's desire to be treated and is believed to improve blood circulation in the treated portions of subcutaneous tissue, thereby increasing the removal of harmful factors released in the tissue, increasing the efficacy of the treatment and the rate of healing.

Device 72 was actually constructed, tested and proved to successfully treat subcutaneous tissue. Specifically, jowls (sagging skin below the chin and jawline) of a human female subject above the age of fifty were treated using a device 72 to transdermally induce ultrasonic vibrations in the jowls with the simultaneous application of vacuum (300 mm Hg in the hollow). After three weekly sessions, each session having a 10-minute duration, the jowls were no longer seen.

### Proximal Channel for Illumination of Skin Apparent Through the Hole of Working Face

In some embodiments, the device is configured to irradiate a skin-surface apparent through the hole of the working face of the sonotrode with radiation, for example, to illuminate with therapeutic light a skin-surface apparent through the hole of the working face of the sonotrode. In some such embodiments that include a proximal channel, the proximal channel is configured to allow passage of a waveguide for the radiation (e.g., a light guide such as an optical fiber for light) into the proximal channel, allowing irradiation of a skin-surface apparent through the hole of the working face with radiation produced from an external radiation source that is guided to the hollow using the waveguide.

A sonotrode of an embodiment of such a device, sonotrode 118 is schematically depicted in side cross section in FIG. 5. Sonotrode 118 is substantially similar to sonotrode 74 of device 72 with a few differences. A first difference is the presence of an optical element, a concave lens 120 in a proximal portion 102 of hollow 88. A second difference is an optical fiber 122 which passes through axial passage 108 in bolt 75 and then through proximal channel (112c, 112b and 112a) so that a distal tip 124 of optical fiber 122 is located in proximal portion 102 of hollow 88 directed at lens 120. A third difference is that sonotrode 118 is devoid of a hold for seating an O-ring.

For use, the device is prepared as usual, including by functionally-associating the transducer with a power supply and connecting optical fiber 122 to a light source (such as a laser known in the art of skin treatment). A lubricant such as mineral oil is applied to the area of skin that is to be treated. Working face 94 is contacted with the surface of skin that is to be treated, with continuous back-and-forth or circular motion as is known in the art of subcutaneous fat treatment.

In a first mode the ultrasound power supply is activated to transdermally treat subcutaneous tissue with ultrasonic vibrations through working face 94.

In a second mode the light source is activated to illuminate the skin surface located in hole 96 of working face 94. Light from the light source is guided by optical fiber 122 to emerge from distal tip 124 to pass through lens 120. Lens 120 causes the light from optical fiber 122 to diverge to illuminate at least some, preferably all, of the skin apparent through hole 96 of working face 94. Any wavelength or combination of wavelengths of light may be used. In some preferred embodiments, light having a wavelength of 1060 nm (e.g., from a light source including a laser configured to produce light having a wavelength of 1060 nm) known for its utility in the transdermal treatment of subcutaneous tissue.

In some embodiments, either the first mode or the second mode are activated. In some embodiments, the first mode and the second mode are alternatingly activated during a single treatment session, e.g., 10 seconds of the first mode and 10 seconds of the second mode. In some embodiments, the two modes are simultaneously activated for at least some of the time of a treatment session.

Embodiment without Evacuation of Air or Illumination with Light

In some embodiments, the device is configured for evacuation of air from the hollow during operation of the device, such as device 72 with sonotrode 74.

In some embodiments, the device is configured for illumination of a skin-surface apparent through the hole of the working face with light, such as a device comprising sonotrode 118.

In some embodiments, the device is configured for transdermal treatment of subcutaneous tissue with ultrasonic vibrations as known in the art of sonotrodes without evacuation of air from the hollow or illumination of skin. A sonotrode 126 of an embodiment of such a device is schematically depicted in side cross section in FIG. 6.

Sonotrode 126 is substantially similar to sonotrodes 74 and 118, with a few differences. Sonotrode 126 is devoid of proximal channels. Instead of an axial bolt 75 with an axial passage 108, sonotrode 126 is associated with a transducer and a reflector with a solid axial bolt 17. Further, inner conical surface 86 and hollow 88 are both complete right cones with a conical apex at the proximal portion 102 of hollow 88.

Additional Embodiment with Evacuation of Air

As noted above, in some embodiments, the device is configured for evacuation of air from the hollow during operation of the device. In some such embodiments, the device comprises a non-axial through-channel through the stem and/or the conical wall. In some embodiments, the through-channel provides fluid communication (e.g., of air) between the hollow and the outside. Alternatively or additionally, in some embodiments, the through-channel provides for the passage of a physical component (e.g., a light guide such as an optical fiber) between the hollow and the outside.

A sonotrode 128 of an embodiment of such a device that is configured for evacuation of air from the hollow through a non-axial through-channel is schematically depicted in side cross section in FIG. 7.

Sonotrode 128 is substantially similar to sonotrode 126, with a few differences. Sonotrode 128 includes a 2 mm diameter non-axial through-channel 130 and a functionally-associated connector 114. Connector 114 is similar to connector 114 of device 72, allowing connection of non-axial through-channel 130 to a suction generator such as a pump.

Operation of a device including sonotrode 128 is substantially identical to operation of device 72 with sonotrode 74 and includes treatment of subcutaneous tissue with ultrasonic vibrations and evacuation of air from the hollow during operation of the device through non-axial through-channel 130.

Embodiment with Evacuation of Air and Illumination of Skin

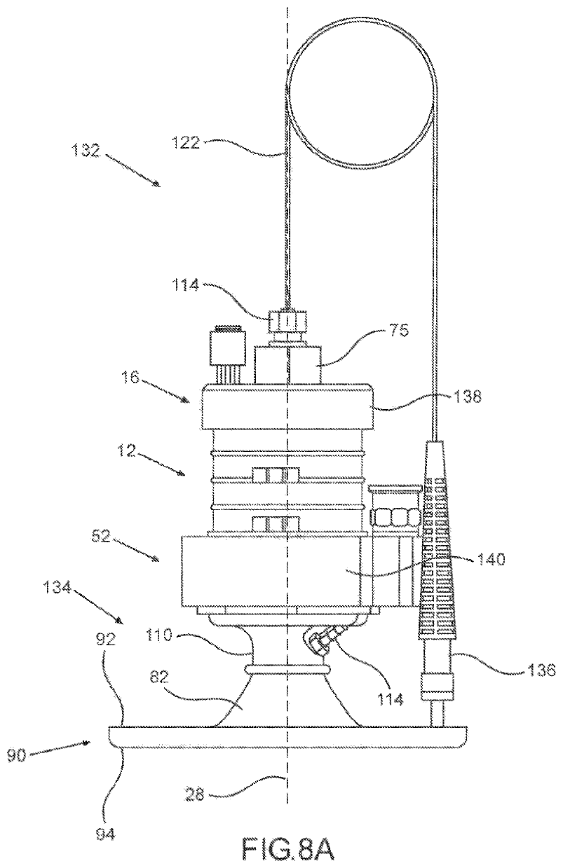

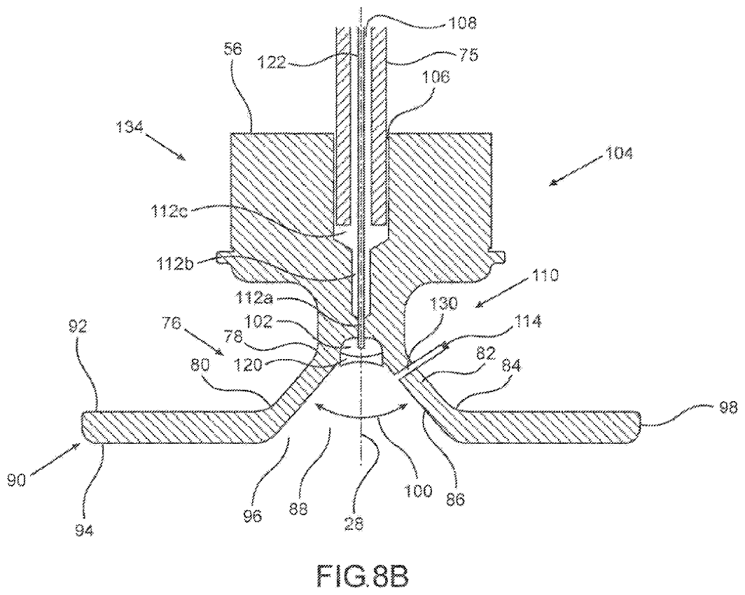

In some embodiments, a device is configured for both illumination with light of a skin-surface apparent through the hole of the working face (similarly to the device comprising sonotrode 118 depicted in FIG. 5) and for evacuation of air from the hollow (similar to device 72 comprising sonotrode 74 depicted in FIG. 4 and the device comprising sonotrode 128 depicted in FIG. 7). An embodiment of such a device, device 132 comprising a sonotrode 134, is schematically depicted in FIG. 8A in side view and sonotrode 134 is depicted in schematic side cross section in FIG. 8B.

As seen in FIG. 8B, sonotrode 134 is substantially similar to sonotrode 118 with the addition of a non-axial through-channel 130 and an adaptor 114 functionally-associated therewith as described for sonotrode 128.

In FIG. 8A additional features of device 132 are seen including a standard connecting component 136 allowing connection of the proximal end of optical fiber 122 with a laser, an upper cooling jacket 138 and a lower cooling jacket 140.

Operation of device 132 is identical to the operation of device 118 with the air evacuation of device 72 and the device comprising sonotrode 128 and is not repeated here for the sake of brevity.

### Further Embodiments Configured for Irradiation of Skin

As noted above, in some embodiments a device according to the teachings herein is configured to irradiate a skin-surface apparent through the hole of the working face of the sonotrode with electromagnetic radiation. The configuration for irradiation is such that the radiation comes from inside the hollow towards the open end of the hollow.

Exemplary such embodiments include: a device comprising sonotrode 118 discussed with reference to FIG. 5 and device 132 discussed with reference to FIGS. 8A and 8B. In such devices an optical fiber 122 passes through an axial passage 108 of an axial bolt 75 and through an axial proximal channel 112 of the sonotrode to that a distal tip of optical fiber 122 is located in proximal portion 102 of hollow 88. Light from a light source which is functionally-associated with a proximal end of optical fiber 122 is guided by optical fiber 122 to emerge from the distal tip of optical fiber 122 towards lens 120. Lens 120 causes the light from the distal tip of optical fiber 122 to diverge in order to illuminate at least some, preferably all, of the skin apparent through hole 96 of working face 94.

In some alternative but similar embodiments, a device is devoid of an optical fiber 122. In some such embodiments, the device resembles a device including a sonotrode 118 or a device 132 as discussed immediately hereinabove. However, instead of an optical fiber 122, a portion of a radiation source (e.g., a laser or the aperture of a laser) is at least partially located inside axial passage 108 of axial bolt 75 and/or axial proximal channel 112. In such embodiments, the radiation source is positioned inside passage 108 and/or channel 112 so that radiation exiting the aperture of the radiation source moves axially towards hole 96 in working face 94 so that when the radiation source is activated, a skin-surface apparent through hole 96 is irradiated with the radiation.

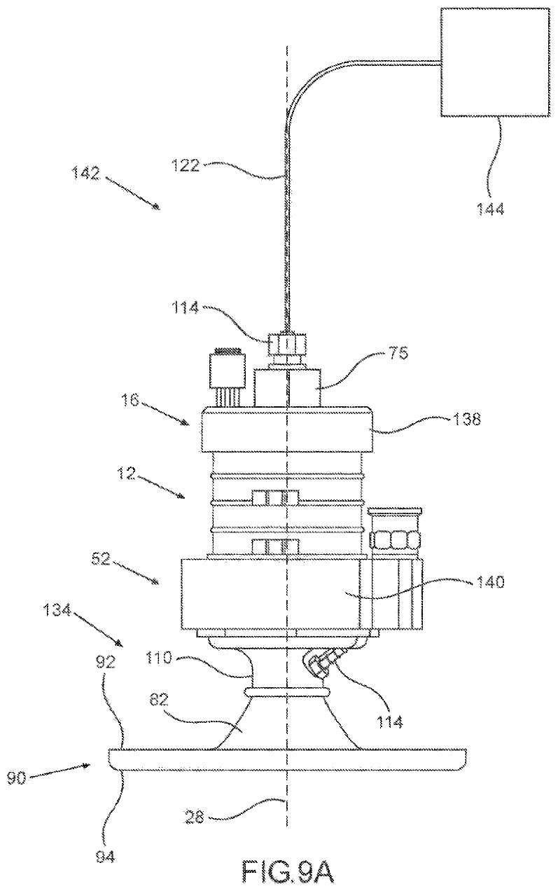

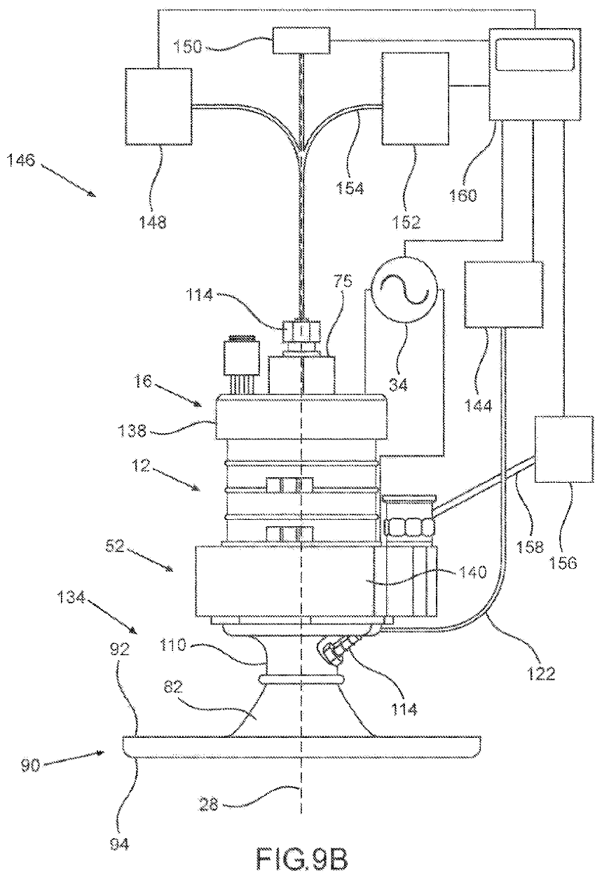

In FIG. 9A, a device 142 similar to device 132 depicted in FIGS. 8A and 8B is schematically depicted. In device 142, component 122 is waveguide to guide radiation generated by a radiation source 144 into a hollow of a sonotrode 134 to irradiate a skin-surface apparent through the hole in a working face 94. In some embodiments, radiation source 144 is a component of device 142. In some alternative embodiments, radiation source 144 is not a component of device 142.

In some embodiments, waveguide 122 is an optical fiber for guiding light (e.g., IR, UV, visible) from light source 144 (e.g., comprising a laser, a diode laser, solid-state laser, a semiconductor laser, a source of non-coherent light, an LED, a flashlamp or an IPL source) to illuminate skin apparent through the hole in working face 142.

In some embodiments, waveguide 122 is a microwave waveguide for guiding microwaves from a microwave source 144 (e.g., comprising a magnetron) to irradiate skin apparent through the hole in working face 142 with microwave radiation. In some such embodiments, there is an optical element analogous to lens 122 (depicted in FIG. 8B), which is an optical element to change the direction of at least some of the microwaves emerging in the hollow from waveguide 122, for example, in some embodiments to ensure that most or all of a skin surface apparent through the hole is simultaneously irradiated.

In some embodiments, waveguide 122 is a terahertz waveguide for guiding terahertz radiation from a terahertz source 144 to irradiate skin apparent through the hole in working face 142 with terahertz radiation. In some such embodiments, there is an optical element analogous to lens 122 (depicted in FIG. 8B), which is an optical element to change the direction of at least some of the terahertz radiation emerging in the hollow from waveguide 122, for example, in some embodiments to ensure that most or all of a skin surface apparent through the hole is simultaneously irradiated.

In FIG. 9B, a device 146 is schematically depicted. Device 146 is similar to device 142 depicted in FIG. 9A with a number of differences. A first difference is that waveguide 122 does not provide axial optical communication with the hollow of the sonotrode through an axial passage and axial proximal channel as in device 142. Instead, in device 146, waveguide 122 is connected to connector 114 to thereby provide optical communication from outside sontrode 134 into the hollow thereof through a non-axial through channel (substantially identical to component 130 depicted in FIG. 8B). Not depicted in FIG. 9B is that the inner surface of the hollow of sonotrode 134 is entirely diffusely-reflective to light or other radiation guided into the hollow by waveguide 122 and that the distal end of waveguide 122 (which is located in the hollow) is functionally associated with an optical component to direct light (that enters the hollow non-axially through waveguide 122) at the hole in working face 94. Components 148, 150, 152 and 154 depicted in FIG. 9B are discussed hereinbelow.

### Ultrasonic Transducer

As noted above, in some embodiments, a device according to the teachings herein comprises an ultrasonic transducer for the generation of ultrasonic longitudinal vibrations, in FIG. 4 ultrasonic transducer 12 where distal face 18 is the radiating surface of ultrasonic transducer 12.

The ultrasonic transducer of a device according to the teachings herein needs to be able to generate sufficiently powerful ultrasonic longitudinal vibrations to allow practice of the teachings herein. If the transducer is not powerful enough, the device will be ineffective while if the transducer is too powerful, a treated subject may be injured.

Accordingly, an ultrasonic transducer of a device according to the teachings herein is an ultrasonic transducer that, during use, is able to have an ultrasonic power output of the selected frequency of a suitable power, in some embodiments an ultrasonic power output of between 40 watts and 120 watts and in some embodiments between 45 watts and 100 watts. That said, it has been found that it is preferable that the ultrasonic transducer have an ultrasonic power output of the selected frequency of between 50 watts and 80 watts, and even of between 60 watts and 70 watts.

Any suitable type of ultrasonic transducer may be used in implementing the teachings herein, for example a prestressed Langevin-type ultrasonic transducer. Suitable such transducers are available from a variety of commercial sources.

### Acoustic Reflector

In some embodiments, a device according to the teachings herein further comprises an acoustic reflector functionally associated with the ultrasonic transducer through the proximal face of the ultrasonic transducer. In FIGS. 4, device 72 comprises an acoustic reflector 16 functionally associated with ultrasonic transducer 12 through proximal face 14. Acoustic reflectors are well-known components in the art commercially available from a variety of sources. Some acoustic reflectors are fluid-filled stainless steel enclosures. In some embodiments such as device 72 depicted in FIGS. 4, an acoustic reflector is configured as a portion of a cooling assembly, e.g., includes a cooling fluid inlet 66 and a cooling fluid outlet 68.

### Ultrasound Power Supply

As known in the art, an alternating current oscillating at an ultrasonic driving frequency is required to drive an ultrasonic transducer to generate ultrasonic vibrations. Such an alternating current is typically provided by an ultrasound power supply functionally associated with the ultrasonic transducer. Accordingly, in some embodiments a device according to the teachings herein comprises an ultrasound power supply functionally associated with the ultrasonic transducer, configured to provide to the ultrasonic transducer, when activated, an alternating current. In FIGS. 4, device 72 comprises an ultrasound power supply 34 functionally associated with ultrasonic transducer 12.

An ultrasound power supply suitable for implementing the teachings herein is preferably configured to provide an alternating current oscillating at a selected ultrasonic frequency for which the sonotrode is configured to operate having sufficient power so that the ultrasonic transducer has a desired power output as discussed above. Accordingly, in some embodiments, the ultrasound power supply is configured to provide an alternating current oscillating at the selected ultrasonic frequency with a power so that the ultrasonic transducer has a power output of between 40 watts and 120 watts, in some embodiments between 45 watts and 100 watts, in some embodiments between 50 watts and 80 watts, and in some embodiments even between 60 watts and 70 watts.

As noted above, the length of a sonotrode and the diameter of the ring portion are at least partially determined by selecting a specific driving frequency and an operating temperature. Specifically, to get the maximum power output, both the length of the sonotrode and the outer diameter of the ring portion of the sonotrode should be close to resonant with the driving frequency: the closer to resonant, the closer to maximum power output.

A sonotrode length of nλ_{longitudinal}/2, where v_{longitudinal} (speed of sound in the sonotrode in the longitudinal direction) is driving frequency *λ_{longitudinal} is resonant with the driving frequency.

A ring portion diameter of nλₜᵣₐₙₛᵥₑᵣₛₑ/2, where vₜᵣₐₙₛᵥₑᵣₛₑ (speed of sound in the sonotrode in the transverse direction) is driving frequency *λₜᵣₐₙₛᵥₑᵣₛₑ is resonant with the driving frequency.

In preferred embodiments, the length and ring portion diameter of a specific sonotrode according to the teachings herein are determined based on the longitudinal speed of sounds and the transverse speed of sound in the material from which the sonotrode is made at a specific temperature (e.g., room temperature or expected operating temperature, e.g., 36°-40° C.).

As is known to a person skilled in the art, the dimensions of an object such as a sonotrode and the speed of sound in a material from which a sonotrode is made change with change in temperature. It has been found that the combined effect of the temperature-dependent changes (dimensions and speed of sound) over the range of temperatures typical for a sonotrode during use are sufficient to significantly reduce the power output of the sonotrode if a single unchanging driving frequency is used during a treatment session.

To overcome this loss of output power, in some embodiments, the ultrasound power supply is configured to provide an alternating current oscillating at a selected ultrasonic frequency that falls within a range of frequencies that the power supply is able to provide.

In some such embodiments, the device and/or power supply and/or controller associated with the device are configured so that an operator can manually select a specific driving frequency that is provided by the power supply that falls within the range of frequencies that the power supply is able to provide. At the beginning and/or during a treatment session, the user can “tune” the driving frequency to be closer to resonant with the sontrode length and ring portion diameter at the moment of tuning, so that the output power is close to the theoretical maximum.

Additionally or alternatively, in some such embodiments, the device and/or power supply and/or controller associated with the device are configured to automatically select a specific driving frequency that is provided by the power supply that falls within the range of frequencies that the power supply is able to provide. At the beginning and/or during a treatment session, the driving frequency is automatically “tuned” to be closer to resonant with the sontrode length and ring portion diameter at the moment of tuning, so that the output power is close to the theoretical maximum.

It has been found that such driving frequency tuning is preferably performed every 2-4 minutes, preferably 2.5-3.5 minutes, e.g., every 3 minutes during a treatment session, allowing adjustment of the driving frequency to account for factors such as the sonotrode temperature that may change during a treatment session.

There is some concern that the temperature-dependent change in longitudinal speed of sound and sontrode length and the temperature-dependent change in transverse speed of sound and sonotrode ring portion diameter would be sufficiently different that it would be impossible to select a single driving frequency that provides an adequate power output at each temperature with the range of the usual operating temperatures of the sontrode. Despite the initial concern, it has been found that for a sonotrode having a specific length and ring portion diameter that are both resonant with the same driving frequency at some temperature between 15° C. and 40° C., it is possible to find a different driving frequency at any temperature between 15° C. to 40° C. that is sufficiently close to resonant with the length and ring portion diameter to provide sufficient power output in both the transverse and longitudinal modes.

### Construction and Material of Sonotrode

A sonotrode of a device according to the teachings herein is made using any suitable method. That said, to avoid imperfections, seams and interfaces that could potentially compromise the vibration-transmission properties of the sonotrode, in some embodiments, all components of the sonotrode is integrally formed.

A sonotrode of a device according to the teachings herein is made of any suitable material. Due to need for low acoustic loss, high dynamic fatigue strength, resistance to cavitation erosion and chemical inertness suitable materials include titanium, titanium alloys, aluminum, aluminum alloys, aluminum bronze or stainless steel. Accordingly, in some embodiments the sonotrode is made of a material selected from the group consisting of titanium, titanium alloys, aluminum, aluminum alloys, aluminum bronze and stainless steel.

Of the listed materials, aluminum and aluminum alloys have an acoustic impedance closest to that of skin, so a sonotrode made of aluminum or aluminum alloys has superior acoustic transmission properties to skin. Accordingly, in some preferred embodiments the sonotrode is made of a material selected from the group consisting of aluminum and aluminum alloys.

In some such embodiments, the working face is coated with aluminum oxide, but such embodiments may leave an aluminum oxide residue on treated skin surfaces so are less preferred. In some embodiments, the working face is coated with an acoustic matching layer (e.g., PVDF or PTFE) on an aluminum oxide layer. Such double layer coating improves the acoustic coupling of the working face with tissue. In such embodiments, the aluminum oxide layer is not more than 75 micrometers thick, not more than 50 micrometers thick, not more than 40 micrometers thick, and even between 5 micrometers and 15 micrometers (e.g., 10 micrometers) while the acoustic matching layer applied to the surface of the aluminum oxide layer (e.g., of PVDF or PTFE) is typically 1 to 50 micrometers thick, preferably 5 to 20 micrometers thick.

In some embodiments where a sonotrode is made of aluminum, a hard anodization layer on the working face may give poor results, apparently the hard anodization layer having an acoustic impedance substantially different from that of skin. In contrast, a soft anodization layer on the working face gives acceptable results. Accordingly, in some embodiments the working face of the sonotrode comprises a soft anodization layer, in some embodiments between 5 and 20 micrometers thick, and in some embodiments, between 8 and 12 micrometers thick, e.g., 10 micrometers thick.

### Cooling Assembly

As is known to a person having ordinary skill in the art, during operation of an ultrasonic transducer an associated sonotrode may be heated to a temperature that makes skin contact with the working face of the sonotrode uncomfortable or even harmful. Additionally, heating of subcutaneous tissue may lead to excessive heating of the skin.

To reduce the incidence of such undesirable effects when the device is used, in some embodiments the device is configured to actively cool at least a portion of the working face. To this end, in some embodiments, a device further comprises a cooling assembly configured, when activated, to cool at least a portion of the working face, directly or indirectly (e.g., by cooling a distal part of the transducer or the sonotrode which is in thermal communication with the working face. In some embodiments, a device further comprises cooling-fluid channels in thermal communication with the working face, e.g., the cooling-fluid channels are in thermal communication with the sonotrode.

During use of the device, such cooling-fluid channels can be functionally associated with an appropriately configured cooling device or cooling assembly that drives a cooling fluid through the cooling-fluid channels, thereby cooling the working face. In some embodiments, the device further comprises a cooling assembly functionally associated with the cooling-fluid channels configured, when activated, to drive a cooling fluid through the cooling-fluid channels, thereby cooling the working face.

Cooling assemblies suitable for use with sonotrodes are well known, see for example, the cooling assembly described in U.S. Pat. No. 9,545,529 of the Applicant, which is included by reference as if fully set forth herein.

Additional Uses of Channels and/or Passages

As discussed above. some devices according to the teachings herein include one or more channels/passages that provide communication from outside of the sonotrode into the hollow, e.g., one or more axial passages and/or one or more non-axial through channels. Such channels are useful for configuring a device for applying suction and/or irradiating a skin-surface apparent through the hole of the working face of the sonotrode with electromagnetic radiation. In some embodiments, such channels or passages are useful for configuring a device according to the teachings herein for additional and/or alternate functionalities.

In some embodiments, a device according to the teachings herein is further configured for acquisition of images of a skin surface apparent through the hole of the working face of the sonotrode from inside the hollow. In some such embodiments, the device further comprises a camera, the camera aperture optically-associated with a passage and/or through-channel in the sonotrode so that when activated, the camera acquires an image of a skin surface apparent through the hole of the working face from inside the hollow. The camera is any suitable camera, in some embodiments the camera is selected from the group consisting of light cameras (e.g., cameras that acquire images of reflected light) and terahertz imaging cameras and scanners (e.g., from TeraSense Group, San Jose, CA USA). In some embodiments, the camera is directly mounted on the sonotrode and associated with a passage and/or through channel without a waveguide so that radiation reflected from a skin surface apparent through the hole of the working face of the sonotrode directly enters the camera aperture through the lens of the camera. Alternatively, in some embodiments, the configuration of the device for image acquisition is that the device comprises a waveguide having a proximal end associable with the aperture of a camera and a distal end of the waveguide leads to inside the hollow of the sonotrode, the waveguide providing optical communication from inside the hollow to the proximal end of the waveguide. In some embodiments, the waveguide passes through a passage and/or through channel. As a result, radiation such as light or terahertz radiation reflected from a skin surface apparent through the hole in the working face of the sonotrode is directed by the waveguide to the aperture of the camera. In some such embodiments, the device further comprises optical elements such as one or more of a prism, a mirror and a lens to direct radiation reflected from a skin surface apparent through the hole in a way that allows for improved image acquisition. In preferred such embodiments, the device is additional configured to irradiate the skin surface apparent through the hole for the purpose of image acquisition.

In some embodiments, a device according to the teachings herein is further configured for determining the temperature of a skin surface apparent through the hole of the working face of the sonotrode from inside the hollow. Any suitable device or component for determining the temperature of a skin surface may be combined or integrated with a device according to the teachings herein to allow determining a skin surface temperature, for example a fiber optic temperature sensor such as available from Advanced Energy Industries, Inc., Denver, CO, USA. Preferably, at least part of such a component or device passes through a passage and/or through channel.

In some embodiments, a device according to the teachings herein is further configured for administration of materials to a skin surface apparent through the hole of the working face of the sonotrode from inside the hollow. Typical materials are medicaments or cosmetics, administered in any suitable form, for example, as a powder, liquid, aerosol or spray. Any suitable device or component for administration of materials to a skin surface apparent through the hole of the working face of the sonotrode from inside the hollow may be combined with or integrated with a device according to the teachings herein.

In some such embodiments, a passage and/or through-channel is functionally associated with a diaphragm. For administration of a material, the tip of a needle is used to pierce the diaphragm and then a desired material is administered through the needle. e.g., with the help of a syringe.

In some such embodiments, a passage and/or through channel is configured to allow passage of or connection to a material-delivery conduit. In some such embodiments, a material-delivery conduit that passes through a passage and/or through channel or that is connected to a passage and/or through channel is a component of the device.

Device 146 depicted in FIG. 9B comprises a camera 148 functionally associated with the hollow of sonotrode 134 through an optical fiber that passes axially through the hollow of sonotrode 134 through an axial passage and axial proximal channel as described above, thereby providing axial optical communication between camera 148 and the hollow of sonotrode 134. When activated, camera 148 acquires images (video or stills) of a skin surface apparent through the hole in working face 94, which images are stored or displayed in real time on a suitable device as known in the art. During image-acquisition by camera 148, a skin surface apparent through the hole in working face 94 is illuminated with light from an LED that is located inside the hollow and receives electrical power through a wire that passes in parallel with the optical fiber associated with camera 148.

Device 146 depicted in FIG. 9B comprises a thermometer 150 functionally associated with the hollow of sonotrode 134 through an optical fiber that passes axially through the hollow of sonotrode 134 through an axial passage and axial proximal channel as described above, thereby providing axial optical communication between thermometer 150 and the hollow of sonotrode 134. When activated, thermometer 150 acquires temperature of a skin surface apparent through the hole in working face 94, which temperature is stored or displayed in real time on a suitable device as known in the art.

Device 146 depicted in FIG. 9B is further configured for administration of materials to a skin surface apparent through the hole of working face 94 from inside the hollow. Specifically, reservoir/pump 152 is functionally associated with the inside of the hollow through a material-delivery conduit 154. When the pump of reservoir/pump 152 is activated, a material such as a liquid medicament is taken from the reservoir of reservoir/pump 152 and forced through conduit 154 which distal end opens out into the hollow. The material is forced out of the distal end of conduit 154 as a spray that is axially-directed but diverges sufficiently to cover most of a skin-surface apparent through the hole in working face 94.

Device 146 depicted in FIG. 9B is functionally-associated with a vacuum pump 156 through a suction conduit 158 through a non-depicted connector that provides fluid communication between conduit 158 and the hollow of sonotrode 134, The connector is located at the back side of device 146 as depicted in FIG. 9B. When vacuum pump 156 is activated, vacuum pump 156 evacuates air from the hollow through conduit 158, so that device 146 can be used to apply suction to skin apparent through the hole in working face 94.

Device 146 depicted in FIG. 9B further comprises a controller 160, a general-purpose computer that is software- and hardware-modified to control operation of device 146. Specifically, controller 160 is configured to allow simultaneous, alternating (e.g., serial, consecutive) and independent operation of all the other components of device 146 in any combination and permutation including:
to activate ultrasound power supply 34 to drive ultrasonic transducer 12;
to activate radiation source 144 to irradiate of a skin-surface apparent through the hole in working face 94 with radiation;
to activate camera 148 to acquire images of a skin-surface apparent through the hole in working face 94;
to activate thermometer 150 to determine the temperature of a skin-surface apparent through the hole in working face 94;
to activate the pump of reservoir/pump 152 to administer a material to a skin-surface apparent through the hole in working face 94; and
to activate vacuum pump 156 to apply suction to a skin-surface through the hole in working face 94.

### Pulsed Ultrasonic Treatment

As discussed in the introduction, in the art it is known to treat tissue using an ultrasonic transducer functionally-associated with a sonotrode. The working face of the sonotrode is acoustically coupled to a surface of tissue and an alternating current (AC) oscillating at an ultrasonic driving frequency is supplied from an ultrasound power supply to drive the ultrasonic transducer. The piezoelectric elements of the ultrasonic transducer expand and relax at the driving frequency in response to the oscillations of the AC potential, thereby generating ultrasonic longitudinal vibrations with the frequency of the driving frequency. The generated ultrasonic longitudinal vibrations propagate axially through the sonotrode to the working face. The working face applies the ultrasonic vibrations to the surface, inducing ultrasonic longitudinal vibrations in the tissue.

In the art, it is known to continuously apply ultrasonic vibrations during a session of treatment of subcutaneous tissue, for example for reducing the amount of subcutaneous fat therein, for at least 10 seconds and typically for 5-20 minutes.

The Inventors herein disclose that superior results, for example for treatment of subcutaneous tissue, for example for reducing the amount of subcutaneous fat therein, are achieved by the periodic application of ultrasonic vibration pulses during a session of treatment of subcutaneous tissue, for example for reducing the amount of subcutaneous fat therein, at a rate of at least 2 pulses per second, each pulse having a duration of less than 250 millisecond and any two pulses separated by at least 10 milliseconds. Without wishing to be held to any one theory, it is currently believed that the beginning of each pulse generates a shockwave in the subcutaneous tissue, which shockwave provides the superior results.

Thus according to an aspect of some embodiments of the teachings herein, there is provided a device for treatment of tissue with ultrasonic vibrations, the device comprising:
i. a sonotrode with a working face;
ii. functionally associated with the sonotrode, an ultrasonic transducer,
iii. functionally associated with the ultrasonic transducer, an ultrasound power supply configured to provide an alternating current (AC) oscillating at an ultrasonic driving frequency to drive the ultrasonic transducer, and
iv. a controller configured to receive a user-command to cause the working face to vibrate at an ultrasonic frequency and, subsequent to receipt of such a command, to activate other components of the device to cause the working face to periodically ultrasonically vibrate at a rate of at least 2 pulses per second, each pulse having a duration of less than 250 millisecond and any two pulses separated by a rest phase of at least 10 milliseconds.

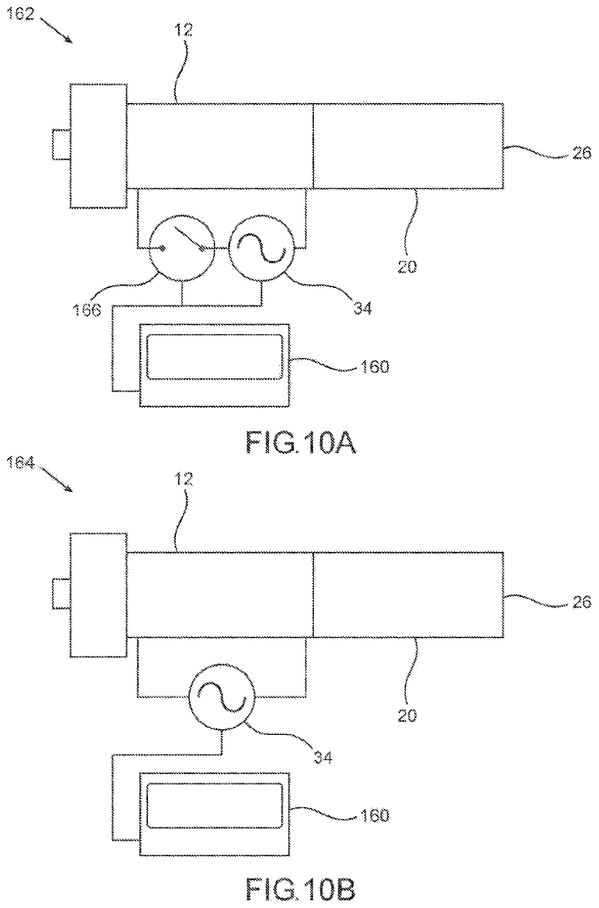

In FIGS. 10A and 10B, two such devices are schematically depicted, device 162 in FIG. 10A and device 164 in FIG. 10B. Both devices include a sonotrode 20 with a working face 26 that is functionally associated with an ultrasonic transducer 12. Ultrasonic transducer 12 is functionally associated with an ultrasound power supply 34. Both devices further comprise a controller 60, a general-purpose computer which is software- and hardware-modified in accordance with the above-listed features.

In some embodiments, the power supply is configured to operate continuously when activated and the device further comprises a controller-controlled switch providing electrical communication between the ultrasonic transducer and the ultrasound power supply, the switch having at least two states:
a closed state where the alternating current provided by the power supply is directed to the ultrasonic transducer to drive the ultrasonic transducer, and
an open state where the alternating current provided by the power supply is not directed to the ultrasonic transducer to drive the ultrasonic transducer,
   and the controller is configured to place the switch in the closed state to provide a pulse and to place the switch in the open state to provide a rest phase.

Device 162 depicted in FIG. 10A comprises a controller-controlled switch 166 having an open state (depicted) and a closed state in accordance with the above-listed features.

Additionally or alternatively, the power supply has at least two states:
an ‘on’ state where the power supply provides the alternating current to drive the ultrasonic transducer, and
an ‘off’ state where the power supply does not provide the alternating current to drive the ultrasonic transducer,
   and the controller is configured to direct the power supply to the on state to provide a pulse and to direct the power supply to the off state to provide a rest phase.

Power supply 34 of device 164 depicted in FIG. 10B has at least two states, an ‘on’ state and an ‘off’ state, and controller 160 is configured to direct the power supply to the on state to provide a pulse and to direct the power supply to the off state to provide a rest phase in accordance with the above-listed features.

As noted above, the device is for treatment of tissue with ultrasonic vibrations. As used herein, the tissue is living tissue of an organism, in preferred embodiments an animal such as a human. In some embodiments, the device is for transdermal treatment of tissue with ultrasonic vibrations and the components of the device are configured for such as known to a person having ordinary skill in the art. In some embodiments, is for transdermal treatment of subcutaneous tissue and the components of the device are configured for such as known to a person having ordinary skill in the art.

The intensity of the pulses is any suitable intensity sufficient to achieve a desired effect. Typically, the intensity is at least 50% of the intensity of an analogous ultrasound treatment using continuous application of ultrasonic vibrations as known in the art.

The sonotrode is any suitable sonotrode, including any suitable sonotrode known in the art. In some embodiments, the sonotrode is any one of the sonotrodes described herein.

The ultrasonic transducer is any suitable ultrasonic transducer, including any suitable ultrasonic transducer known in the art. In some embodiments, the ultrasonic transducer is any one of the ultrasonic transducers described herein.

The ultrasound power supply is any suitable ultrasound power supply, including any suitable ultrasound power supply known in the art that is suitable for use with the selected transducer and sonotrode.

As noted above, the controller is configured to cause the working face to periodically ultrasonically vibrate at a rate of at least 2 pulses per second, each pulse having a duration of less than 250 millisecond and any two pulses separated by a rest phase of at least 10 milliseconds.

The ratio of the duration of a pulse to the duration of a rest phase is any suitable ratio. In some embodiments, during a second of operation, the ratio is between 30% pulse/70% rest phase to 70% pulse/30% rest phase.

In some embodiments, during a second of operation, the ratio is between 30% pulse/70% rest phase to 70% pulse/30% rest phase. in some embodiments between 30% pulse/70% rest phase to 60% pulse/40% rest phase and in some embodiments even 30% pulse/70% rest phase to 50% pulse/50% rest phase. In some preferred embodiments, during a second of operation, the ratio is between 35% pulse/65% rest phase to 45% pulse/55% rest phase, preferably between 37% pulse/63% rest phase to 43% pulse/57% rest phase, e.g., 40% pulse/60% rest phase.

The waveform (i.e., intensity as a function time) of the driving alternating current (AC) provided by the ultrasound power supply is any suitable waveform. In preferred embodiments, the waveform is a square wave.

The frequency of the pulses is any suitable frequency, as noted above, being at least 2 pulses per second (2 Hz). In some embodiments, the frequency of the pulses is not more than 20 Hz and even not more than 15 Hz. In some embodiments, the frequency of the pulses is not less than 3 Hz and even not less than 4 Hz. In some preferred embodiments, the frequency of the pulses is not less than about 5 Hz and not more than about 15 Hz. In some preferred embodiments, the frequency of the pulses is selected from the group of about 5 Hz, about 10 Hz and about 15 Hz.

The rise time of the driving current at the transducer is any suitable rise time (for a given pulse, the time from 0 current at the transducer to the maximum current). Generally speaking, shorter rise times are preferred. In some embodiments, the rise time is not more than about 10% of a pulse width, not more than about 8% and even not more than about 5% of the pulse width.

In some embodiments, a controller of a device is configured to allow pulsed application of ultrasonic vibrations (described above) alternating with continuous application of ultrasonic vibrations (as known in the art). In some such embodiments, a treatment duration with pulsed ultrasound application is between about 5 to about 60 seconds alternating with a treatment duration with continuous ultrasound application of between about 5 to about 60 seconds. In some embodiments, both treatment durations are between about 10 and about 30 seconds, e.g., about 15 to about 25 seconds.

According to an aspect of some embodiments of the teachings herein, there is also provided a method for treatment of tissue with ultrasonic vibrations, the method comprising:
acoustically coupling working face of a sonotrode with a tissue surface;
for a treatment duration, causing the working face to periodically vibrate at an ultrasonic frequency at a rate of at least 2 pulses (of ultrasonic vibrations) per second,
each pulse having a duration of less than 250 millisecond and any two pulses separated by a rest phase of at least 10 milliseconds,
   wherein the intensity of the pulses and the treatment duration are sufficient to achieve a desired result.

The tissue surface is any tissue surface. In some embodiments, the tissue surface is skin, especially human skin.

The method is for treatment of tissue with ultrasonic vibrations. As used herein, the tissue is living tissue of an organism, in preferred embodiments an animal such as a human. In some embodiments, the method is for transdermal treatment of tissue with ultrasonic vibrations. In some embodiments, the method is for transdermal treatment of subcutaneous tissue. In some embodiments, the method is for the transdermal reduction in the volume of subcutaneous fat so that the intensity of the pulses and the treatment duration are sufficient to achieve a reduction of the volume of subcutaneous fat underlying the surface.

The intensity of the pulses is any suitable intensity sufficient to achieve a desired effect. Typically, the intensity is at least 50% of the intensity of an analogous ultrasound treatment using continuous application of ultrasonic vibrations as known in the art.

The treatment duration is any suitable treatment duration. In some embodiments, the treatment duration is at least 50% of the duration of an analogous ultrasound treatment using continuous application of ultrasonic vibrations as known in the art. Typically, the duration is between about 1 minute to about 1 hour.

Any suitable device or combination of devices, especially a device according to the teachings herein, may be used for implementing an embodiment of the method. In some embodiments a known devices such as known devices for transdermal treatment of subcutaneous fat may be used for implementing an of the method. In some embodiments, a known device is software-modified for implementing an embodiment of the method.

In some embodiments of the method, the ratio of the duration of a pulse to the duration of a rest phase is any suitable ratio. In some embodiments, during a second of operation, the ratio is between 30% pulse/70% rest phase to 70% pulse/30% rest phase.

In some embodiments of the method, during a second the ratio is between 30% pulse/70% rest phase to 70% pulse/30% rest phase. in some embodiments between 30% pulse/70% rest phase to 60% pulse/40% rest phase and in some embodiments even 30% pulse/70% rest phase to 50% pulse/50% rest phase. In some preferred embodiments, during a second, the ratio is between 35% pulse/65% rest phase to 45% pulse/55% rest phase, preferably between 37% pulse/63% rest phase to 43% pulse/57% rest phase, e.g., 40% pulse/60% rest phase.

The frequency of the pulses is any suitable frequency, as noted above, being at least 2 pulses per second (2 Hz). In some embodiments, the frequency of the pulses is not more than 20 Hz and even not more than 15 Hz. In some embodiments, the frequency of the pulses is not less than 3 Hz and even not less than 4 Hz. In some preferred embodiments, the frequency of the pulses is not less than about 5 Hz and not more than about 10 Hz.

The rise time of the driving current at the transducer is any suitable rise time (for a given pulse, the time from 0 current at the transducer to the maximum current). Generally speaking, shorter rise times are preferred. In some embodiments, the rise time is not more than about 10% of a pulse width, not more than about 8% and even not more than about 5% of the pulse width.

A given treatment session is typically between about 5 and about 30 minutes. That said, any treatment longer than 25 minutes, and even longer than 20 minutes can be tedious and tiring for the person performing the treatment, especially when suction is applied to the skin. Accordingly, a treatment session is typically between 5 and 20 minutes.

In some embodiments, pulsed application of ultrasonic vibrations (described above) is alternated with continuous application of ultrasonic vibrations (as known in the art) during a single treatment session. In some such embodiments, a treatment duration with pulsed ultrasound application is between about 5 to about 60 seconds alternating with a treatment duration with continuous ultrasound application of between about 5 to about 60 seconds. In some embodiments, both treatment durations are between about 10 and about 30 seconds, e.g., about 15 to about 25 seconds.

In the description above, was described that in some embodiments, one or more of various components are in communication with the inside of the hollow of the sonotrode including a radiation source, a camera, a thermometer, an administration component such as reservoir/pump 152 and a suction component such as vacuum pump 156. Although not all options and permutations have been depicted herein for the sake of brevity and clarity, it is clear to a person having ordinary skill in the art upon perusal of the description herein, none, some or all of such components present are in axial communication with the hollow, e.g., through an axial channel in an axial bolt and, additionally or alternatively, none, some or all of such components present are in non-axial communication with the hollow, e.g., through a non-axial through.

Unless otherwise defined, all technical and scientific terms used herein have the same meaning as commonly understood by one of ordinary skill in the art to which the invention pertains. In case of conflict, the specification, including definitions, takes precedence.

As used herein, the terms “comprising”, “including”, “having” and grammatical variants thereof are to be taken as specifying the stated features, integers, steps or components but do not preclude the addition of one or more additional features, integers, steps, components or groups thereof. As used herein, the indefinite articles “a” and “an” mean “at least one” or “one or more” unless the context clearly dictates otherwise.

As used herein, when a numerical value is preceded by the term “about”, the term “about” is intended to indicate +/−10%. As used herein, a phrase in the form “A and/or B” means a selection from the group consisting of (A), (B) or (A and B). As used herein, a phrase in the form “at least one of A, B and C” means a selection from the group consisting of (A), (B), (C), (A and B), (A and C), (B and C) or (A and B and C).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements. Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

Section headings are used herein to ease understanding of the specification and should not be construed as necessarily limiting.

1. A device suitable for treating subcutaneous tissue, comprising:
a. an ultrasonic transducer for generation of ultrasonic vibrations having a proximal face and a distal face; and
b. a sonotrode with a sonotrode axis including:
i. a proximal face in contact with, and acoustically-coupled to, said distal face of said ultrasonic transducer,
ii. a conical portion having a smaller-radius proximal end and a larger-radius distal end, wherein said conical portion is defined by a conical wall having an outer conical surface and an inner conical surface, which inner conical surface at least partially defines a hollow, and
iii. a ring portion extending radially outwards from said distal end of said conical portion having a ring-shaped proximal face and a ring-shaped distal face which is a working face of sonotrode, a hole of said working face constituting an open end of said hollow.

2. The device of claim 1, wherein said sonotrode is configured for a selected ultrasonic frequency, to act as an acoustic amplitude transformer for both longitudinal and transverse vibration.

3. The device of claim 2, wherein said configuration to operate at a selected ultrasonic driving frequency is that the length of the sonotrode from the proximal face to the working face is:
nλ_{longitudinal}/2
where n is a positive integer; and
λ_{longitudinal} is the wavelength of ultrasonic longitudinal waves in the sonotrode,

4. The device of claim 1, configured to apply suction to a skin-surface through said hole of said working face of said sonotrode.

5. The device of claim 1, configured to irradiate a skin-surface apparent through said hole of said working face of said sonotrode with electromagnetic radiation.

6. The device of claim 1, wherein said ultrasonic transducer is a prestressed Langevin-type transducer including an axial bolt having a distal end and a proximal end.

7. The device of claim 6, wherein said axial bolt includes an axial passage between said distal end and said proximal end of said axial bolt.

8. The device of claim 1, wherein the diameter of said hole is between 10% and 70% of the diameter of said ring portion.

9. The device of claim 1, said sonotrode further comprising a stem, said stem having a proximal face that is said proximal face of said sonotrode and a distal end which is said proximal end of said conical wall.

10. The device of claim 1, said sonotrode comprising a proximal channel between said hollow and outside of said sonotrode near said transducer.

11. The device of claim 10, wherein said ultrasonic transducer is a prestressed Langevin-type transducer including an axial bolt having an axial passage between a distal end and a proximal end of said axial bolt, and
said sonotrode comprising a bore for engaging said distal end of said axial bolt,
so that said proximal channel of said sonotrode and said axial passage of said axial bolt together provide communication between said hollow and said proximal end of said axial bolt.

12. The device of claim 1, said sonotrode comprising a non-axial through-channel providing communication between said hollow and outside of said sonotrode through said conical wall.

13. The device of claim 1, wherein said ultrasonic transducer is configured to operate at a driving frequency of between 30 kHz and 200 kHz.

14. The device of claim 1, further comprising an ultrasound power supply to drive the ultrasonic transducer at a driving frequency of between 30 kHz and 200 kHz.

15. A device suitable for treating subcutaneous tissue, comprising:
a. an ultrasonic transducer for generation of ultrasonic vibrations having a proximal face and a distal face; and
b. a sonotrode with a sonotrode axis including:
i. a proximal face in contact with, and acoustically-coupled to, said distal face of said ultrasonic transducer,
ii. in said sonotrode, an open-ended hollow,
ii. a distal face, said distal face being a working face of said sonotrode, a hole of said working face constituting an open end of said hollow,
the device configured to irradiate a skin-surface apparent through said hole of said working face of said sonotrode with electromagnetic radiation.

16. The device of claim 15, the device further comprising a waveguide in optical communication with said hollow so that activation of a radiation source that is functionally associated with said waveguide leads to irradiation of a skin surface apparent through said hole of said working face of said sonotrode with electromagnetic radiation from the radiation source.

17. The device of claim 15, the device further comprising at least one optical element to refract the electromagnetic radiation.

18. The device of claim 17, said optical element is configured to refract the electromagnetic radiation in order to:
direct at least some of the radiation towards said open end of said hollow;
direct at least some of the radiation away from an inner surface of said hollow; or
distribute the radiation in a desired manner at said open end of said hollow.

19. A device suitable for treating subcutaneous tissue, comprising:
a. an ultrasonic transducer for generation of ultrasonic vibrations having a proximal face and a distal face; and
b. a sonotrode with a sonotrode axis including:
i. a proximal face in contact with, and acoustically-coupled to, said distal face of said ultrasonic transducer,
ii. in said sonotrode, an open-ended hollow,
ii. a distal face, said distal face being a working face of said sonotrode, a hole of said working face constituting an open end of said hollow,
wherein said ultrasonic transducer is a prestressed Langevin-type transducer including an axial bolt having a distal end and a proximal end, and an axial passage between said distal end and said proximal end of said axial bolt.

20. The device of claim 19, wherein said ultrasonic transducer is prestressed at between 45 N/m and 100 N/m.
